(19) United States
(12) Patent Application Publication
(10) Pub. No.: US 2018/0273571 A1
(43) Pub. Date: Sep. 27, 2018

(54) CONCISE SYNTHESIS OF UREA DERIVATIVES OF AMPHOTERICIN B

(71) Applicant: THE BOARD OF TRUSTEES OF THE UNIVERSITY OF ILLINOIS, Urbana, IL (US)

(72) Inventors: Martin D. BURKE, Champaign, IL (US); Stephen DAVIS, Westfield, IN (US)

(21) Appl. No.: 15/541,747

(22) PCT Filed: Jan. 8, 2016
(86) PCT No.: PCT/US2016/012602

§ 371 Date: Jul. 6, 2017

Related U.S. Application Data
(60) Provisional application No. 62/100,988, filed on Jan. 8, 2015.

(51) Int. Cl. C07H 17/08; A01N 47/18; A01N 47/36; A01N 47/42; A01N 43/90

(52) U.S. Cl. CPC C07H 17/08; A01N 47/18; A01N 43/90; A01N 47/42; A01N 47/36

(57) ABSTRACT

Provided are certain derivatives of amphotericin B (AmB) characterized by reduced toxicity and retained anti-fungal activity. Certain of the derivatives are C16 urea derivatives and C16 carbamate derivatives of AmB. Also provided are methods of making the AmB derivatives.

### RELATED APPLICATION

This application claims the benefit of priority to U.S. Provisional Patent Application Ser. No. 62/100,988, filed Jan. 8, 2015.

### GOVERNMENT SUPPORT

This invention was made with Government support under Grant No. GM080436, awarded by the National Institutes of Health. The government has certain rights in the invention.

### BACKGROUND OF THE INVENTION

For more than half a century amphotericin B (AmB) has served as the gold standard for treating systemic fungal infections. AmB has a broad spectrum of activity, is fungicidal, and is effective even against fungal strains that are resistant to multiple other agents. Surprisingly, clinically significant microbial resistance has remained exceptionally rare while resistance to next generation antifungals has appeared within just a few years of their clinical introduction. Unfortunately, AmB is also highly toxic. Thus, the effective treatment of systemic fungal infections with AmB is all too often precluded, not by a lack of efficacy, but by dose-limiting side effects. Some progress has been made using liposome delivery systems, but these treatments are prohibitively expensive and significant toxicities remain. Thus, a less toxic, but equally effective AmB derivative stands to have a major impact on human health.

### SUMMARY OF THE INVENTION

An aspect of the invention is a compound represented by formula (I) or a pharmaceutically acceptable salt thereof,

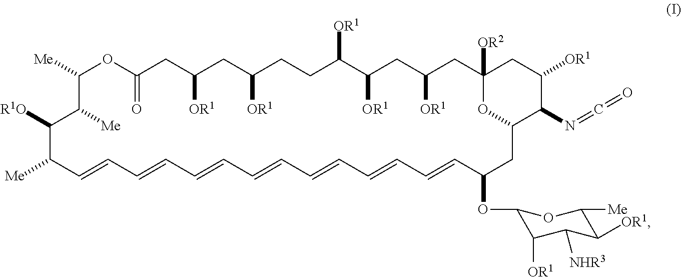

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

An aspect of the invention is a compound represented by formula (II) or a pharmaceutically acceptable salt thereof,

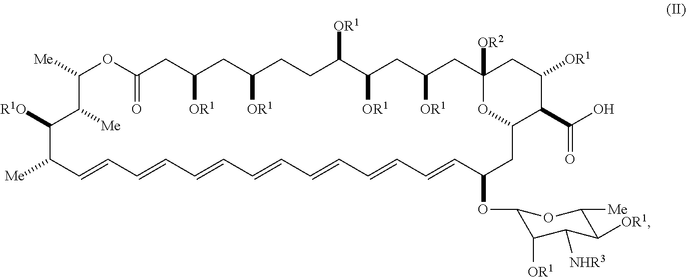

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

An aspect of the invention is a compound represented by formula (III) or a pharmaceutically acceptable salt thereof,

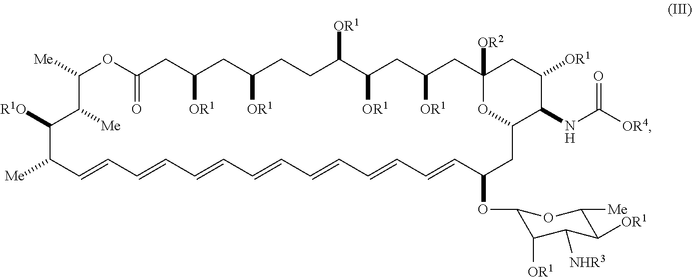

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—; and
R⁴ represents alkyl, aralkyl, alkenyl, aryl, or cycloalkyl.

An aspect of the invention is AmBCU-allylester or a pharmaceutically acceptable salt thereof,

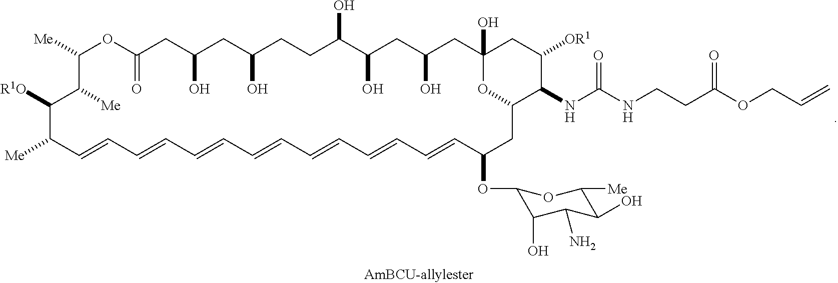

An aspect of the invention is a method of preparing a compound of formula (IV), or a pharmaceutically acceptable salt thereof, comprising the step of combining a compound of formula (I) or a pharmaceutically acceptable salt thereof, and a compound represented by R⁵-XH or R⁵-X-M, thereby producing a compound of formula (IV), wherein the compound of formula (IV) is represented by:

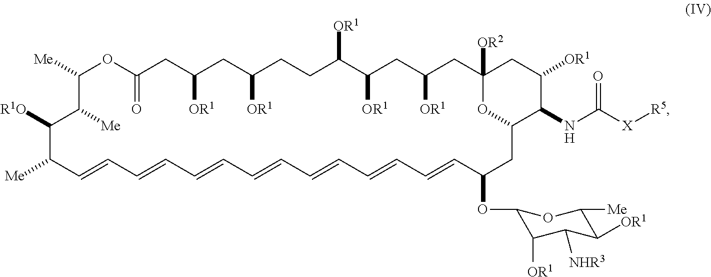

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—;
X represents O, NH, or N(R⁶); and
R⁵ and R⁶ each independently represent alkyl, aralkyl, alkenyl, aryl, or cycloalkyl;
   and
M is an alkali metal cation, an alkaline earth cation, or a transition metal cation.

An aspect of the invention is a method of making a compound of formula (IV) as disclosed in the specification and drawings.

### BRIEF DESCRIPTION OF THE DRAWINGS

FIG. 1 depicts structural formulas of AmB and certain derivatives thereof.

FIG. 2A depicts a general synthetic scheme for the synthesis of AmB urea derivatives from a minimally protected intermediate 1.

FIG. 2B depicts a three-step syntheses of AmBMU and AmBAU from AmB, and a four-step synthesis of AmBCU from AmB.

FIG. 3 depicts a number of synthetic schemes for preparing C16 amino AmB derivatives by reacting oxazolidinone 2 with any of a wide range of heteroatom nucleophiles.

FIG. 4 depicts an alternate synthesis of AmB ureas and carbamates. Protection of the C15 alcohol with a TES group allows for isolation of isocyanate 5. Lewis acid-mediated carbamate formation is possible from 5.

FIG. 5 is a group of three graphs depicting kidney fungal load (colony forming units, cfu) in neutropenic mice inoculated intravenously with C. albicans and then treated two hours later with a single intraperitoneal dose of vehicle control, AmB, AmBMU, or AmBAU. Panel A, 1 mg/kg AmB, AmBMU, or AmBAU. Panel B, 4 mg/kg AmB, AmBMU, or AmBAU. Panel C, 16 mg/kg AmB, AmBMU, or AmBAU.

FIG. 6 is a graph depicting lethality in healthy mice of single intravenous administration in the doses indicated of AmB, AmBMU, or AmBAU.

### DETAILED DESCRIPTION

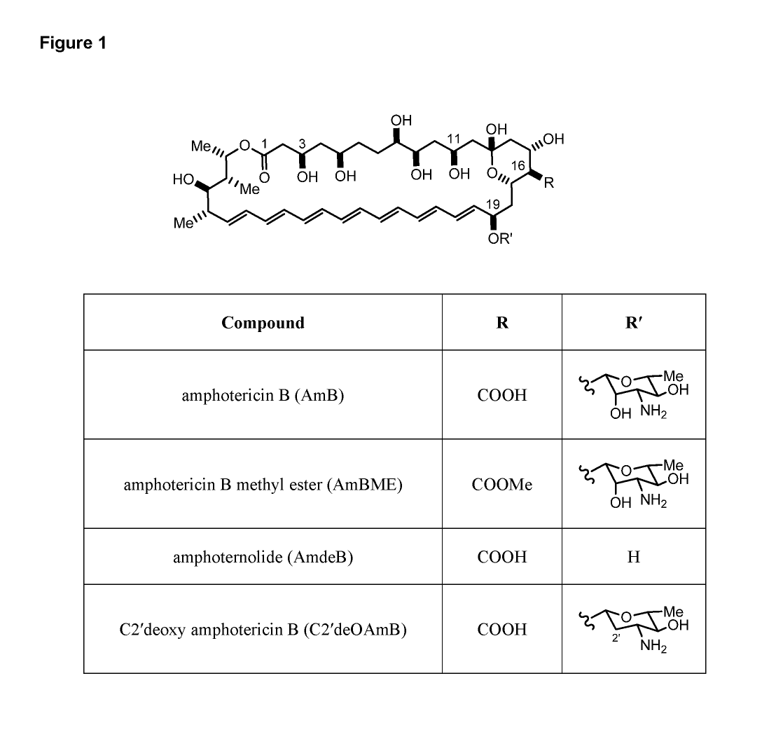

A lack of understanding of the mechanism(s) by which AmB is toxic to yeast and human cells has thus far hindered the rational development of a clinically successful derivative. The longstanding accepted mechanism of action of AmB has been ion channel formation within a cell's membrane leading to electrochemical gradient disruption and eventually cell death. This model suggests that development of a less toxic derivative requires selective ion channel formation in yeast versus human cells. Contrary to this longstanding model, our group recently discovered that the primary mechanism of action of AmB is not ion channel formation, but simple ergosterol binding. Yeast and human cells possess different sterols, ergosterol and cholesterol, respectively. Therefore, the new model suggests a simpler and more actionable roadmap to an improved therapeutic index; i.e., a less toxic AmB derivative would retain potent ergosterol binding capability, but lack the ability to bind cholesterol. A derivative was recently reported in which removal of the C2′ hydroxyl group from the mycosamine sugar produced a derivative, C2′deOAmB (FIG. 1), which surprisingly retains ergosterol-binding ability, but shows no binding to cholesterol. Wilcock, B C et al., J Am Chem Soc 135:8488 (2013). Consistent with the preferential sterol binding hypothesis, in vitro studies demonstrated that C2′deOAmB is toxic to yeast, but not human cells.

To explain why removal of the C2′ alcohol results in loss of cholesterol binding ability, while maintaining efficient ergosterol binding, we hypothesized that the AmB structure exists in a ground state conformation capable of binding both sterols. Removal of the C2′ alcohol potentially results in a conformational change of the AmB structure which retains ergosterol binding ability but is incapable of binding cholesterol. A generic molecule is capable of binding two different ligands in a common binding site. Modification at a site distal to the binding pocket alters the binding site conformation. This principle of allosteric modification causes preferential binding of one ligand over the other. Such ligand-selective allosteric effects have not been previously observed in small molecule-small molecule interactions. Encouragingly, ligand selective allosteric modifications have been observed in proteins which bind multiple ligands in a common binding site. It was hypothesized that removal of the C2′ alcohol allosterically modifies the sterol binding pocket, accounting for the decrease in cholesterol binding ability.

A previously obtained X-ray crystal structure of N-iodoacyl AmB suggested a prominent water bridged hydrogen bond joining the C2′ alcohol to the C13 hemiketal. If such a water bridged hydrogen bond helped rigidify the ground state conformation of AmB, it would follow that removal of the C2′ alcohol abolishes this interaction and thereby could potentially enable adoption of an alternative ground state conformers having altered affinities for cholesterol and ergosterol. This crystal structure may represent the ground state conformation of AmB which is capable of binding both ergosterol and cholesterol. The present invention is based at least in part on the discovery of rigidifying features observed in the crystal structure of N-iodoacyl AmB. The invention described herein explores disruption or removal of such rigidifying features in order to access alternative ground state conformations, thereby altering the AmB sterol binding profile. Three additional intramolecular rigidifying features were identified with the potential of stabilizing the AmB ground state: 1) a salt bridge between the C41 carboxylate and C3′ ammonium, 2) a 1,3,5 hydrogen bonding network between C1 carbonyl O, C3 and C5 alcohols, and 3) a 1,3,5 hydrogen bonding network between the C9, C11, and C13 alcohols.

### New Allosteric Site: C41-C3′ Carboxylate

The salt bridge interaction is the energetically strongest of the proposed rigidifying features. Thus, systematic modification of the group appended to the C16 carbon was targeted as the first series of derivatives to further probe this allosteric modification model. Multiple AmB derivatives modifying the C41 carboxylate have been reported including esters and amides among others. However, all previous AmB derivatives maintain a carbon atom appended to the C16 carbon.

The present invention is based at least in part on the discovery that appending a heteroatom to the C16 carbon has a great impact on the salt bridge interaction. Therefore, an efficient, chemoselective synthetic strategy was sought out in order to gain access to such a derivative. Complicating such a goal, AmB possesses a dense array of complex and sensitive functional groups, making the direct synthesis of derivatives difficult.

One possible route to the AmB derivative with heteroatom substitution at C16 is a three-step synthesis including Fmoc protection, methyl ketal formation, and Curtius rearrangement (e.g., promoted by diphenyl phosphoryl azide). This synthetic plan provides an intermediate isocyanate which is trapped intramolecularly to generate oxazolidinone 2 (Scheme 1).

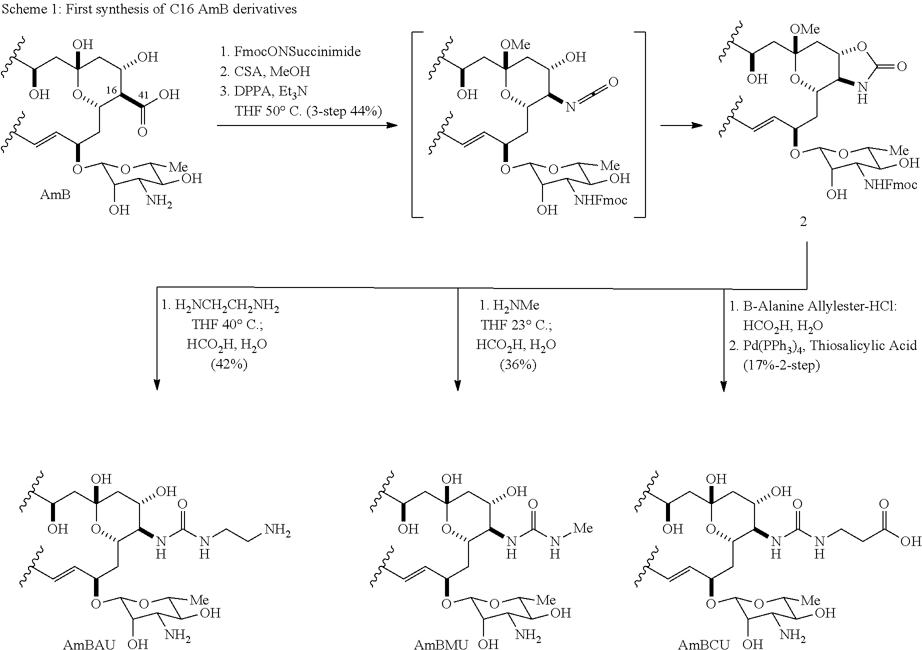

Treatment of a minimally protected variant of AmB with diphenyl phosphoryl azide (DPPA) cleanly promotes a stereospecific Curtius rearrangement in which the C16-C41 bond is cleaved and the resulting isocyanate is intramolecularly trapped by the neighboring C15 alcohol to form an oxazolidinone 2. This particular oxazolidinone, in turn, is surprisingly reactive to ring-opening with primary amines under mild conditions to yield a new class of urea containing amphotericins (AmBAU, AmBMU, and AmBCU) having a C16-nitrogen bond. Interestingly, the parent heterocycle, 2-oxazolidinone, is unreactive under the same conditions.

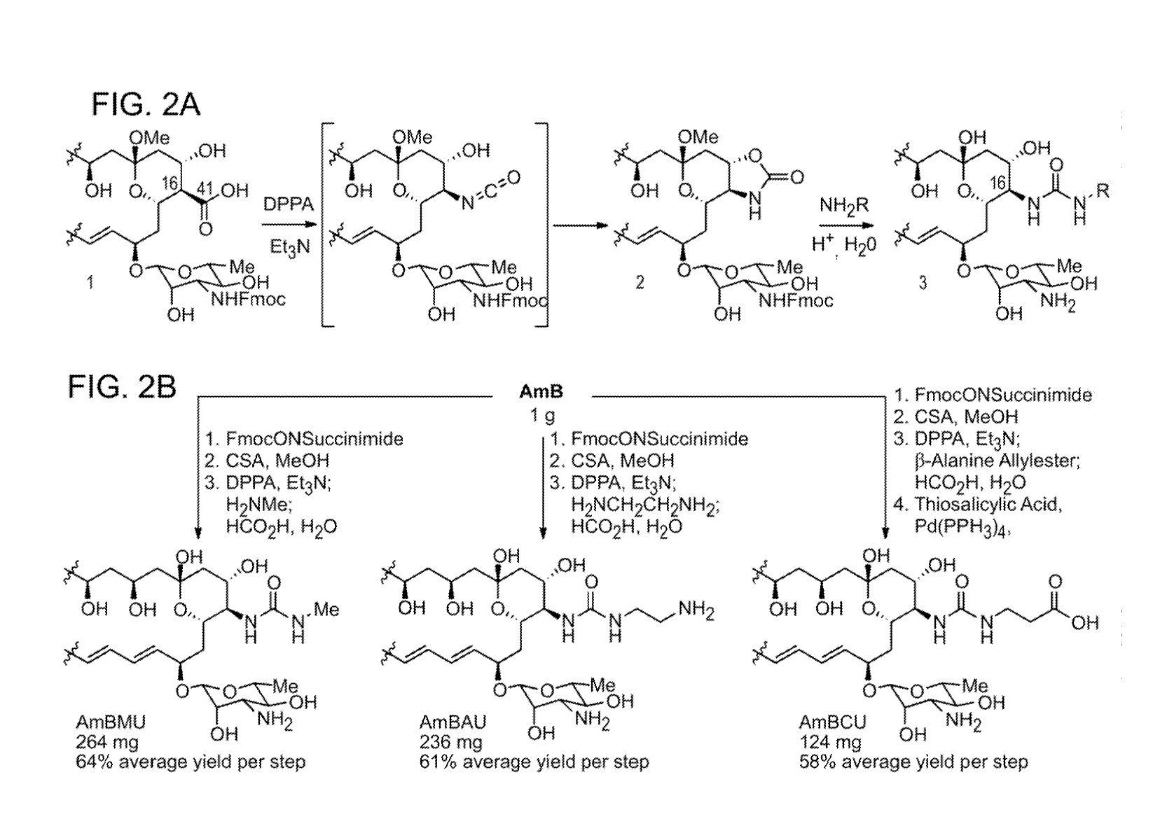

Minimally protected AmB derivative 1 can be directly converted to 3 in a scalable one-pot operation involving serial addition of diphenyl phosphoryl azide (DPPA), an amine, and aqueous acid (FIG. 2B). Starting with 1 g of fermented AmB and using methyl amine as the nucleophile, this overall three-step sequence yields 264 mg of AmB methyl urea (AmBMU). Employing ethylene diamine produces 236 mg of AmB amino urea (AmBAU), and in a four step-variant, reaction with β-alanine allylester followed by deallylation yields 124 mg AmB carboxylatoethyl urea (AmBCU). This chemistry thus provides rapid, efficient, and scalable access to these new derivatives starting with the natural product that is already fermented on the metric ton scale.

With efficient access to this novel AmB chemotype, ureas AmBAU, AmBMU, and AmBCU were compared to AmB and a range of previously reported AmB derivatives in an in vitro antifungal and human cell toxicity screen. Yeast toxicity was measured with broth microdilution assays (MIC) against Saccharomyces cerevisiae. Human cell toxicity was studied by determining the amount of compound required to cause 90% hemolysis of human erythrocytes (EH₉₀). These results are summarized in Table 1. Amphotericin B inhibits S. cerevisiae growth at 0.5 μM while 90% red blood cell lysis occurs at only 10.4 μM. Removal of mycosamine (AmdeB) completely abolishes cell-killing activity in both yeast and human cell assays. Methyl esterification (AmBME) retains antifungal activity at 0.25 μM against S. cerevisiae, while decreasing hemolysis concentration to one third that seen with AmB. C41MethylAmB shows, similar to AmBME, an MIC of 0.5 μM while causing hemolysis at 22.0 μM. As previously observed, simple amidation to form amino amide AmB derivative AmBAA or methyl amide AmBMA increased potency against yeast to 0.03 μM and 0.25 μM, respectively. Hemolysis activity remained similar to AmBME and C41MeAmB. Bis-amino alkylated amide derivative AmBNR₂ was previously shown to moderately improve the therapeutic index. Consistent with precedent, AmBNR₂ shows increased antifungal activity compared to AmB, while requiring elevated concentrations to cause hemolysis at 48.5 μM.

TABLE 1
In vitro biological activity of AmB derivatives

| | | MIC (μM) | EH90 (μM) |
|---|---|---|---|
| Name | Compound | S. cerevisiae | red blood cells |
| AmB | 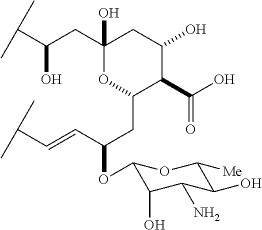 | 0.5 | 10.37 ± 1.17 |
| AmdeB | 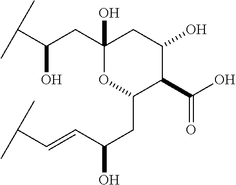 | >500 | >500 |
| AmBME | 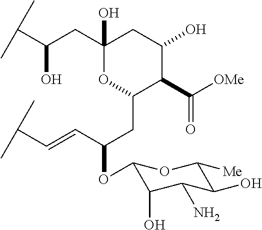 | 0.25 | 30.67 ± 5.38 |
| C41MeAmB | 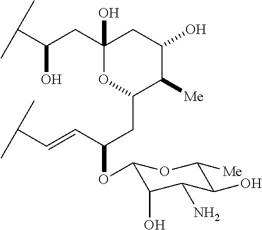 | 0.5 | 22.03 ± 6.26 |
| AmBAA | 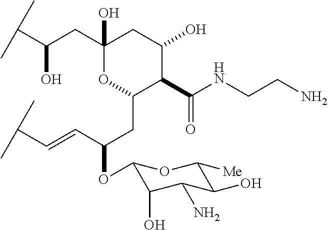 | 0.03 | 33.96 ± 8.85 |
| AmBMA | 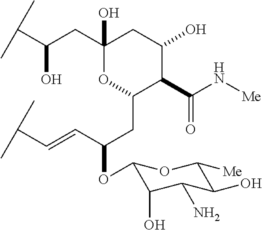 | 0.25 | 15.32 ± 3.39 |
| AmBNR₂ | 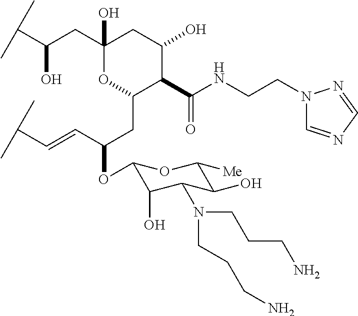 | 0.25 | 48.5 ± 8.7 |
| AmBMU | 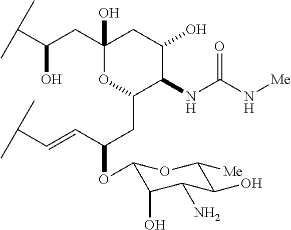 | 0.25 | >500 |
| AmBAU | 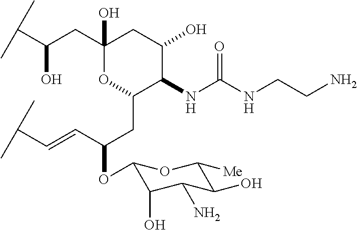 | 0.125 | >500 |
| AmBCU | 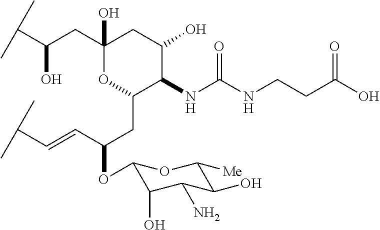 | 3 | 323.8 ± 30.2 |

Urea derivatives AmBAU, AmBMU, and AmBCU maintain potent antifungal activity ranging from 0.125 μM to 3 μM against S. cerevisiae. Surprisingly, AmBAU, AmBMU, and AmBCU possessed drastically decreased toxicity towards red blood cells. AmBMU and AmBAU did not reach an EH₉₀ even at 500 μM, greater than 45× that observed with AmB. AmBCU required 324 μM to cause 90% hemolysis in red blood cells, more than 30× required by AmB. Encouraged by this initial therapeutic index screen the urea series was further tested against the clinically relevant fungal cell line Candida albicans. C. albicans is the most common human fungal infection. AmB inhibits yeast grown of C. albicans at 0.25 μM. Similar to the trend seen with S. cerevisiae, the potency of the urea derivatives increased with increasing amount of cationic character. AmBAU, AmBMU, and AmBCU require 0.25, 0.5, and 1 μM respectively (Table 2).

TABLE 2
In vitro antifungal activity of AmB urea
derivatives against C. albicans

| Compound | AmB | AmBMU | AmBAU | AmBCU |
|---|---|---|---|---|
| MIC (μM) | 0.25 | 0.5 | 0.25 | 1 |

Following the allosteric modification model, ureas AmBAU, AmBMU, and AmBCU maintain potent ergosterol binding ability, but have lost the ability to bind cholesterol.

### Synthetic Route to AmB Ureas and Other Derivatives

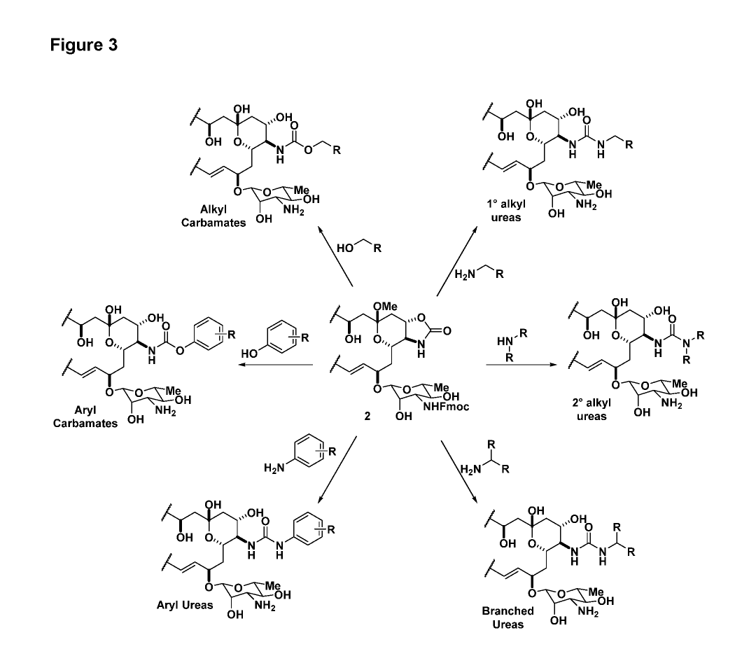

Because the amphotericin B (AmB) urea derivatives AmBAU, AmBMU, and AmBCU all showed no detectable binding to cholesterol and dramatic decreases in toxicity, it is reasonable to expect that additional AmB urea derivatives will share these critical features. A small subset of the possible accessible derivatives is outlined in FIG. 3. Oxazolidinone 2 could be intercepted with primary amines to generate primary ureas, secondary amines to generate secondary ureas, and primary amines with alpha branching to create ureas with stereochemistry introduced at the alpha position. Additionally, oxazolidinone 2 could be opened with anilines to create aryl ureas, phenols to create aryl carbamates, or alcohols to generate alkyl carbamates. Thus, there is substantial opportunity for extensive optimization of the pharmacological properties of this new family of less toxic amphotericins. Certain nucleophilic additions to the oxazolidinone may pose a synthetic challenge, due in part to the reactivity of the nucleophile or the oxazolidinone electrophile. Thus, it would be beneficial to intercept a more reactive electrophile that could grant access to an even wider family of AmB analogs. For example, upon Curtius rearrangement of 1 an isocyanate is presumably generated (Scheme 1, FIG. 2A). Such an isocyanate would be a promising reactive intermediate for diversification.

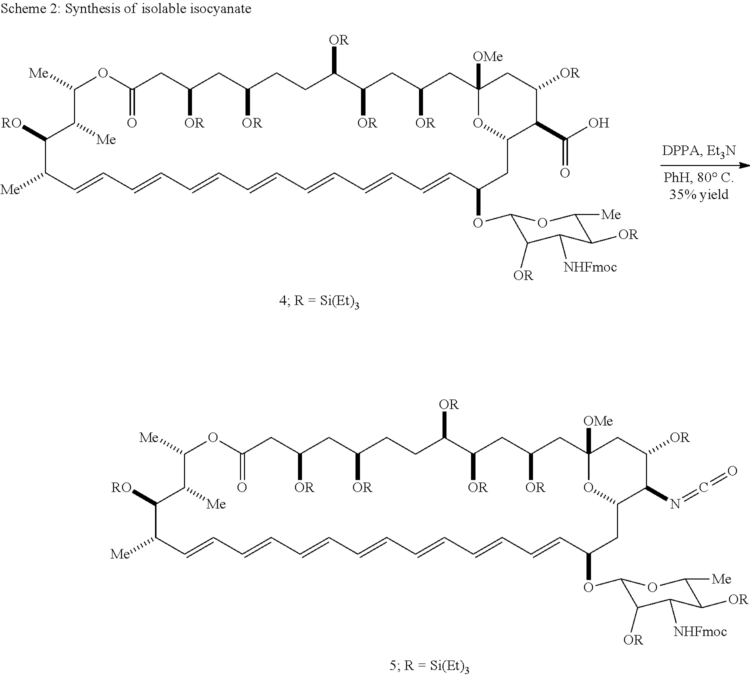

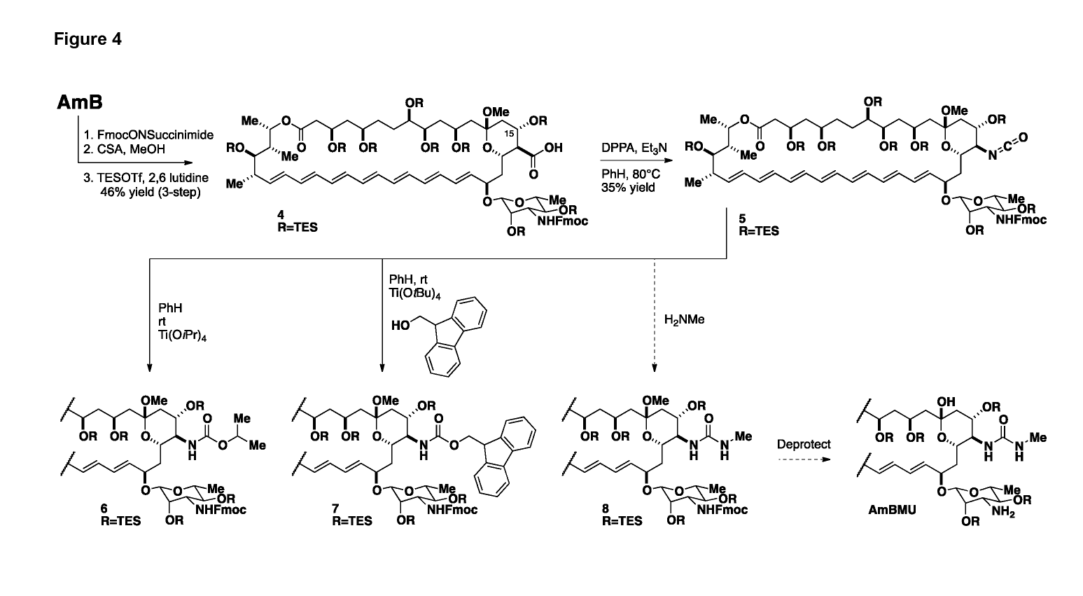

Formation of an isolable isocyanate is achievable when the C15 hydroxyl group is protected. An exemplary reaction is shown in Scheme 2, where starting with AmB, a 3-step protection sequence involving Fmoc carbamate formation, methyl ketal formation, and global silylation forms persilyl AmB 4. Exposure of 4 to DPPA at elevated temperatures effects a Curtius rearrangement. Because the neighboring C15 alcohol is protected, the desired isocyanate 5 can be isolated. Isocyanate 5, similar to oxazolidinone 2, is a versatile intermediate that can be intercepted to form a variety of AmB derivatives (FIG. 4). Exposure of 5 to the Lewis acid titanium isopropoxide transfers an isopropoxy group to the isocyanate forming isopropyl carbamate 6. Similarly, ligand exchange between titanium tertbutoxide with Fmoc alcohol facilitates the formation of Fmoc carbamate 7. Furthermore, this sequence provides an alternate route to synthesize AmB ureas. For example exposure of 5 to methyl amine followed by deprotection would complete an alternative synthesis of AmBMU.

Exemplary protecting groups that can be used to protect the C15 hydroxyl group include silyl groups (e.g., trialkylsilyl groups, dialkylarylsilyl groups, alkyldiarylsilyl groups, and triaryl silyl groups), and (hydrocarbyloxy)methyl ethers (e.g., (alkoxy)methyl ethers, (alkenyloxy)methyl ethers, (aralkoxy)methyl ethers, (aryloxy)methyl ethers, and ((trialkylsilyl)alkoxy)methyl ethers). More particular examples of such protecting groups include, without limitation, methoxymethyl ether, (phenyldimethylsilyl)methoxymethyl ether, benzyloxymethyl ether, para-methoxybenzyloxymethyl ether, para-nitrobenzyloxymethyl ether, tert-butoxymethyl ether, siloxymethyl ether, 2-methoxyethoxymethyl ether, 2,2,2-trichloroethoxymethyl ether, 2-(trimethylsilyl)ethoxymethyl ether, tetrahydropyranyl ether, trimethylsilyl ether, triethylsilyl ether, triisopropylsilyl ether, diethylisopropylsilyl ether, dimethylhexylsilyl ether, tert-butyldimethylsilyl ether, tert-butyldiphenylsilyl ether, triphenylsilyl ether, and diphenylmethylsilyl ether.

Nucleophiles that can be added to the isocyanate electrophile include alcohols and their corresponding alkoxides, thiols and their corresponding thiolates, and amines.

Examples of amines include, without limitation, 1-(1-Naphthyl)ethylamine; 1-(2-Naphthyl)ethylamine; 1-(4-Bromophenyl)ethylamine; 1,1-Diphenyl-2-aminopropane; 1,2,2-Triphenylethylamine; 1,2,3,4-Tetrahydro-1-naphthylamine; 1,2-Bis(2-hydroxyphenyl)ethylenediamine; 1-Amino-2-benzyloxycyclopentane; 1-Aminoindane; 1-Benzyl-2,2-diphenylethylamine; 1-Cyclopropylethylamine; 1-Phenylbutylamine; 2-(3-Chloro-2,2-dimethyl-propionylamino)-3-methylbutanol; 2-(Dibenzylamino)propionaldehyde; 2,2-Dimethyl-5-methylamino-4-phenyl-1,3-dioxane; 2-Amino-1-fluoro-4-methyl-1,1-diphenylpentane; 2-Amino-3,3-dimethyl-1,1-diphenylbutane; 2-Amino-3-methyl-1,1-diphenylbutane; 2-Amino-3-methylbutane; 2-Amino-4-methyl-1,1-diphenylpentane; 2-Aminoheptane; 2-Aminohexane; 2-Aminononane; 2-Aminooctane; 2-Chloro-6-fluorobenzylamine; 2-Methoxy-α-methylbenzylamine; 2-Methyl-1-butylamine; 2-Methylbutylamine; 3,3-Dimethyl-2-butylamine; 3,4-Dimethoxy-α-methylbenzylamine; 3-Amino-2-(hydroxymethyl)propionic acid; 3-Bromo-α-methylbenzylamine; 3-Chloro-α-methylbenzylamine; 4-Chloro-α-methylbenzylamine; 4-Cyclohexene-1,2-diamine; 4-Fluoro-α-methylbenzylamine; 4-Methoxy-α-methylbenzylamine; 7-Amino-5,6,7,8-tetrahydro-2-naphthol; Bis[1-phenylethyl]amine; Bornylamine; cis-2-Aminocyclopentanol hydrochloride; cis-Myrtanylamine; cis-N-Boc-2-aminocyclopentanol; Isopinocampheylamine; L-Allysine ethylene acetal; Methyl 3-aminobutyrate p-toluenesulfonate salt; N,N′-Dimethyl-1,1′-binaphthyldiamine; N,N-Dimethyl-1-(1-naphthyl)ethylamine; N,N-Dimethyl-1-phenylethylamine; N,α-Dimethylbenzylamine; N-allyl-α-methylbenzylamine; N-Benzyl-α-methylbenzylamine; sec-Butylamine; trans-2-(Aminomethyl)cyclohexanol; trans-2-Amino-1,2-dihydro-1-naphthol hydrochloride; trans-2-Benzyloxycyclohexylamine; α,4-Dimethylbenzylamine; α-Ethylbenzylamine; α-Methylbenzylamine; and β-Methylphenethylamine.

Examples of alcohols include, without limitation, methanol, ethanol, 2-butoxyethanol, propanol, allyl alcohol, methallyl alcohol, prenol, isopropanol, 2,2-dimethylpropan-1-ol, 2-methyl-2-phenylpropan-1-ol, butanol, isobutanol, sec-butanol, tert-butanol, 2-buten-1-ol, pentanol, 2-cyclopenten-1-ol, 4-cyclopenten-1-ol, cyclopentanol, 3-cyclopenten-1-ol, hexanol, cyclohexanol, 3-cyclohexen-1-ol, phenol, 1-naphthol, 2-naphthol, benzyl alcohol, menthol, 1,2-ethanediol, 9-fluorenylmethanol, resorcinol, meta-cresol, cinnamyl alcohol, and geraniol. It should be understood that the alkoxide corresponding to an alcohol can also be used as a nucleophile in a reaction with the isocyanate.

### Compounds of the Invention

An aspect of the invention is a compound represented by formula (I) or a pharmaceutically acceptable salt thereof,

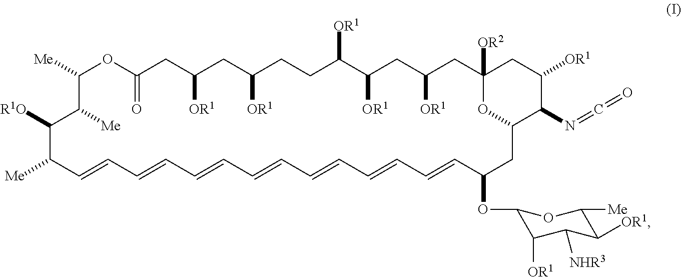

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

In certain embodiments, in the compound of formula (I), R¹ represents trialkylsilyl, for example, triethylsilyl.

In certain embodiments, R² represents methyl.

In certain embodiments, R³ represents (aralkyl)OC(O)—, for example (9-fluorenylmethyl)OC(O)— (i.e., Fmoc).

An aspect of the invention is a compound represented by formula (II) or a pharmaceutically acceptable salt thereof,

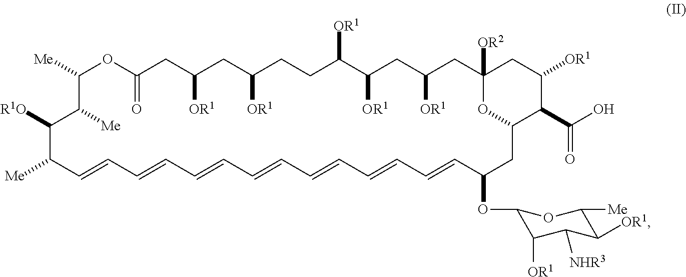

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

In certain embodiments, in the compound of formula (II), R¹ represents trialkylsilyl, for example, triethylsilyl.

In certain embodiments, R² represents methyl.

In certain embodiments, R³ represents (aralkyl)OC(O)—, for example (9-fluorenylmethyl)OC(O)— (i.e., Fmoc).

An aspect of the invention is a compound represented by formula (III) or a pharmaceutically acceptable salt thereof,

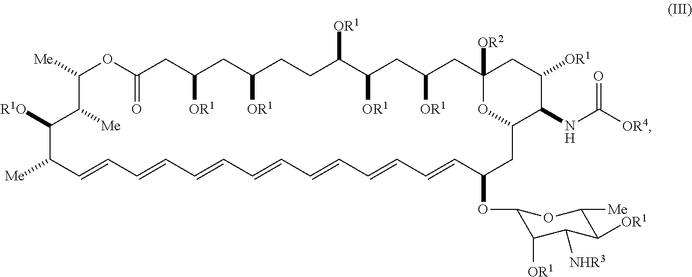

wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—; and
R⁴ represents alkyl, aralkyl, alkenyl, aryl, or cycloalkyl.

In certain embodiments, in the compound of formula (III), R¹ represents trialkylsilyl, for example, triethylsilyl.

In certain embodiments, R² represents methyl.

In certain embodiments, R³ represents (aralkyl)OC(O)—, for example (9-fluorenylmethyl)OC(O)— (i.e., Fmoc).

An aspect of the invention is AmBCU-allylester or a pharmaceutically acceptable salt thereof,

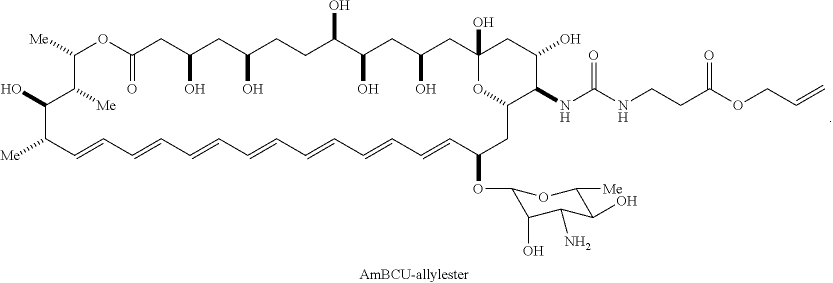

### Methods of Making AmB Derivatives

An aspect of the invention is a method of making a compound of formula (IV), or a pharmaceutically acceptable salt thereof,

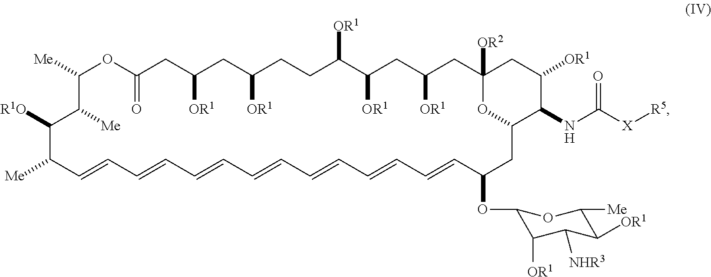

comprising the step of combining a compound of formula (I) or a pharmaceutically acceptable salt thereof, and a compound represented by R⁵-XH or R⁵-X-M, thereby producing the compound of formula (IV);
wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—;
X represents O, NH, or N(R⁶);
R⁵ and R⁶ each independently represent alkyl, aralkyl, alkenyl, aryl, or cycloalkyl;

the compound of formula (I) is represented by:

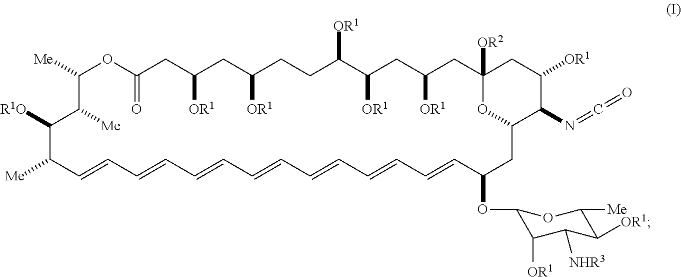

and
M is an alkali metal cation, an alkaline earth cation, or a transition metal cation.

In certain embodiments, the compound of formula (I) or a pharmaceutically acceptable salt thereof is combined with a compound represented by R⁵-XH, wherein the compound represented by R⁵-XH is a primary amine, a secondary amine, or an alcohol.

In certain embodiments, the compound of formula (I) or a pharmaceutically acceptable salt thereof is combined with a compound represented by R⁵-X-M, wherein the compound represented by R⁵-X-M is a metal amide, such as sodium amide, potassium amide, or lithium diisopropylamide. In certain embodiments, the compound represented by R⁵-X-M is a metal alkoxide such as sodium isopropoxide, potassium methoxide, or titanium tertbutoxide. In certain embodiments, M is Na, K, or Ti(O(hydrocarbyl))₃, for example, Ti(OR⁵)₃.

In certain embodiments, the method of making the compound of formula (IV) further comprises the step of combining a compound of formula (II) and a phosphoryl azide, thereby producing a compound of formula (I). The compound of formula (II) is represented by:

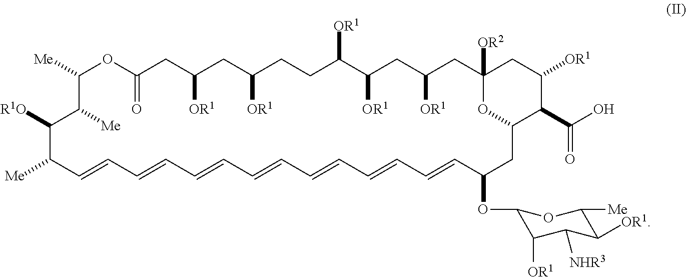

In certain embodiments, the phosphoryl azide is dipheylphosphoryl azide.

In certain embodiments, the step of combining a compound of formula (I) or a pharmaceutically acceptable salt thereof and a compound represented by R⁵-XH or R⁵-X-M further comprises a Lewis acid, for example, a titanium alkoxide.

In certain embodiments, the step of combining a compound of formula (I) or a pharmaceutically acceptable salt thereof and a compound represented by R⁵-XH or R⁵-X-M further comprises solvent such as a polar aprotic solvent or a non-polar solvent.

In certain embodiments, the step of combining a compound of formula (II) and a phosphoryl azide further comprises a Bronsted base. In certain embodiments, the Bronsted base is a tertiary amine.

In certain embodiments, the step of combining a compound of formula (II) and a phosphoryl azide further comprises a solvent such as a polar aprotic solvent or a non-polar solvent.

In certain embodiments of the method, R¹ represents trialkylsilyl, for example, triethylsilyl.

In certain embodiments, R² represents methyl.

In certain embodiments, R³ represents (aralkyl)OC(O)—, for example (9-fluorenylmethyl)OC(O)— (i.e., Fmoc).

Definitions

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, about 6, or about 7 carbons in the ring structure.

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (i.e., an aromatic or heteroaromatic group).

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The term “heteroatom” is art-recognized and refers to an atom of any element other than carbon or hydrogen. Illustrative heteroatoms include boron, nitrogen, oxygen, phosphorus, sulfur and selenium.

The term “nitro” is art-recognized and refers to —NO₂.

The term “halogen” is art-recognized and refers to —F, —Cl, —Br or —I.

The term “sulfhydryl” is art-recognized and refers to —SH.

The term “hydroxyl” is art-recognized and refers —OH.

The term “sulfonyl” is art-recognized and refers to —SO₂⁻.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The terms “amine” and “amino” are art-recognized and refer to both unsubstituted and substituted amines, e.g., a moiety that may be represented by the general formulas:

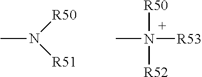

wherein R50, R51 and R52 each independently represent a hydrogen, an alkyl, an alkenyl, —(CH₂)ₘ-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “amido” is art recognized as an amino-substituted carbonyl and includes a moiety that may be represented by the general formula:

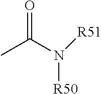

wherein R50 and R51 are as defined above. Certain embodiments of the amide in the present invention will not include imides which may be unstable.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like.

A “polar protic solvent” as used herein is a solvent having a dipole moment of about 1.4 to 4.0 D, and comprising a chemical moiety that participates in hydrogen bonding, such as an O—H bond or an N—H bond. Exemplary polar protic solvents include methanol, ethanol, n-propanol, isopropanol, n-butanol, isobutanol, ammonia, water, and acetic acid.

A “polar aprotic solvent” as used herein means a solvent having a dipole moment of about 1.4 to 4.0 D that lacks a hydrogen bonding group such as O—H or N—H. Exemplary polar aprotic solvents include acetone, N,N-dimethylformamide, acetonitrile, ethyl acetate, dichloromethane, tetrahydrofuran, and dimethylsulfoxide.

A “non-polar solvent” as used herein means a solvent having a low dialectric constant (<5) and low dipole moment of about 0.0 to about 1.2. Exemplary nonpolar solvents include pentane, hexane, cyclohexane, benzene, toluene, chloroform, and diethyl ether.

For purposes of this invention, the chemical elements are identified in accordance with the Periodic Table of the Elements, CAS version, Handbook of Chemistry and Physics, 67th Ed., 1986-87, inside cover.

Also provided are pharmaceutical compositions comprising a compound of the invention, or a pharmaceutically acceptable salt thereof, and a pharmaceutically acceptable carrier. Also provided is a method for making such pharmaceutical compositions. The method comprises placing a compound of the invention, or a pharmaceutically acceptable salt thereof, in a pharmaceutically acceptable carrier.

Compounds of the invention and pharmaceutical compositions of the invention are useful for inhibiting the growth of a fungus. In one embodiment, an effective amount of a compound of the invention is contacted with a fungus, thereby inhibiting growth of the fungus. In one embodiment, a compound of the invention, or a pharmaceutically acceptable salt thereof, is added to or included in tissue culture medium.

Compounds of the invention and pharmaceutical compositions of the invention are useful for the treatment of fungal infections in a subject. In one embodiment, a therapeutically effective amount of a compound of the invention, or a pharmaceutically acceptable salt thereof, is administered to a subject in need thereof, thereby treating the fungal infection.

A fungus is a eukaryotic organism classified in the kingdom Fungi. Fungi include yeasts, molds, and larger organisms including mushrooms. Yeasts and molds are of clinical relevance as infectious agents.

Yeasts are eukaryotic organisms classified in the kingdom Fungi. Yeasts are typically described as budding forms of fungi. Of particular importance in connection with the invention are species of yeast that can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic yeasts include, without limitation, various species of the genus Candida, as well as of Cryptococcus. Of particular note among pathogenic yeasts of the genus Candida are C. albicans, C. tropicalis, C. stellatoidea, C. glabrata, C. krusei, C. parapsilosis, C. guilliermondii, C. viswanathii, and C. lusitaniae. The genus Cryptococcus specifically includes Cryptococcus neoformans. Yeast can cause infections of mucosal membranes, for example oral, esophageal, and vaginal infections in humans, as well as infections of bone, blood, urogenital tract, and central nervous system. This list is exemplary and is not limiting in any way.

A number of fungi (apart from yeast) can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic fungi (apart from yeast) include, without limitation, species of Aspergillus, Rhizopus, Mucor, Histoplasma, Coccidioides, Blastomyces, Trichophyton, Microsporum, and Epidermophyton. Of particular note among the foregoing are A. fumigatus, A. flavus, A. niger, H. capsulatum, C. immitis, and B. dermatitidis. Fungi can cause systemic and deep tissue infections in lung, bone, blood, urogenital tract, and central nervous system, to name a few. Some fungi are responsible for infections of the skin and nails.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, or 95 percent (%) compared to control.

As used herein, the terms “treat” and “treating” refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease.

A “fungal infection” as used herein refers to an infection in or of a subject with a fungus as defined herein. In one embodiment the term “fungal infection” includes a yeast infection. A “yeast infection” as used herein refers to an infection in or of a subject with a yeast as defined herein.

As used herein, a “subject” refers to a living mammal. In various embodiments a subject is a non-human mammal, including, without limitation, a mouse, rat, hamster, guinea pig, rabbit, sheep, goat, cat, dog, pig, horse, cow, or non-human primate. In one embodiment a subject is a human.

As used herein, a “subject having a yeast or fungal infection” refers to a subject that exhibits at least one objective manifestation of a yeast or fungal infection. In one embodiment a subject having a yeast or fungal infection is a subject that has been diagnosed as having a yeast or fungal infection and is in need of treatment thereof. Methods of diagnosing a yeast or fungal infection are well known and need not be described here in any detail.

As used herein, “administering” has its usual meaning and encompasses administering by any suitable route of administration, including, without limitation, intravenous, intramuscular, intraperitoneal, intrathecal, intraocular (e.g., intravitreal), subcutaneous, direct injection (for example, into a tumor), mucosal, inhalation, oral, and topical.

In one embodiment, the administration is intravenous.

In one embodiment, the administration is oral.

As used herein, the phrase “effective amount” refers to any amount that is sufficient to achieve a desired biological effect.

As used herein, the phrase “therapeutically effective amount” refers to an amount that is sufficient to achieve a desired therapeutic effect, e.g., to treat a yeast or fungal infection.

Compounds of the invention can be combined with other therapeutic agents. The compound of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compound of the invention, when the administration of the other therapeutic agents and the compound of the invention is temporally separated. The separation in time between the administration of these compounds may be a matter of minutes or it may be longer.

Examples of other therapeutic agents include other antifungal agents, including AmB, as well as other antibiotics, anti-viral agents, anti-inflammatory agents, immunosuppressive agents, and anti-cancer agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be, for human subjects, from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

In one embodiment, intravenous administration of a compound of the invention may typically be from 0.1 mg/kg/day to 20 mg/kg/day. Intravenous dosing thus may be similar to, or advantageously, may exceed maximal tolerated doses of AmB.

For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Amphotericin B is commercially available in a number of formulations, including deoxycholate-based formulations and lipid-based (including liposomal) formulations. Amphotericin B derivative compounds of the invention similarly may be formulated, for example, and without limitation, as deoxycholate-based formulations and lipid-based (including liposomal) formulations.

For use in therapy, an effective amount of the compound of the invention can be administered to a subject by any mode that delivers the compound of the invention to the desired surface. Administering the pharmaceutical composition of the present invention may be accomplished by any means known to the skilled artisan. Routes of administration include but are not limited to oral, intravenous, intramuscular, intraperitoneal, subcutaneous, direct injection (for example, into a tumor or abscess), mucosal, inhalation, and topical.

For oral administration, the compounds (i.e., compounds of the invention, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds of the invention (or derivatives thereof). The compound of the invention (or derivative) is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (α-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al. (incorporated by reference).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of compound of the invention (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise compound of the invention (or derivative) dissolved in water at a concentration of about 0.1 to 25 mg of biologically active compound of the invention per mL of solution. The formulation may also include a buffer and a simple sugar (e.g., for compound of the invention stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R., Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

### EXAMPLES

Having now described the present invention in detail, the same will be more clearly understood by reference to the following examples, which are included herewith for purposes of illustration only and are not intended to be limiting of the invention.

### Example 1

### Synthesis of AmBMU from Minimally Protected AmB

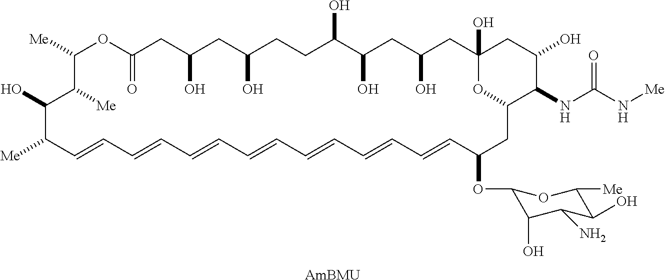

A round bottom flask was charged with amphotericin B (1 g, ca. 1.082 mmol, 1 equiv.) and Fmoc-succinimide (0.55 g, 1.62 mmol, 1.5 equiv.) which were dissolved in a 2:1 mixture of DMF:MeOH (33.8 mL) at room temperature. Pyridine (0.5 mL, 6.21 mmol, 5.74 equiv.) was subsequently added and the reaction was stirred for 12 hours at room temperature. The reaction mixture was then poured into diethyl ether (1.0 L). After stirring for 30 minutes, the resulting yellow precipitate was isolated via Büchner filtration using Whatman #50 filter paper to afford a yellow solid. The filter cake was dried on the filter for 10 minutes and then stored under vacuum for one hour.

The resulting powder was dissolved in 1:1 THF:MeOH (35 mL) and cooled to 0° C. To this solution was added camphorsulfonic acid (138 mg, 0.59 mmol, 0.55 equiv.) and the resulting mixture was stirred for 1 hour at 0° C. The reaction was then quenched at 0° C. with triethylamine (0.14 mL, 0.59 mmol, 0.55 equiv.). The reaction was concentrated in vacuo removing approximately half of the solvent. The resulting saturated solution was poured into 1:1 hexanes:diethyl ether (1.0 L) and the yellow precipitate was collected via Büchner filtration using Whatman #50 filter paper and washed with diethyl ether (200 mL) to yield a yellow solid.

The resulting solid was dissolved in THF (54 mL, 0.02 M). To this solution was added triethylamine (0.15 mL, 1.08 mmol, 1 equiv.) and then diphenyl phosphoryl azide (0.70 mL, 3.25 mmol, 3 equiv.). The reaction was heated to 50° C. and stirred for 12 hours. After 12 hours the reaction was cooled to room temperature and methylamine (2.0M in THF, 4.33 mL, 8.8 mmol, 8 equiv.) was added. The reaction then stirred at room temperature for 8 hours, slowly evolving a yellow precipitate. The reaction mixture was then poured into diethyl ether (1.0 L), and the resulting yellow precipitate was isolated via Büchner filtration using Whatman #50 filter paper to afford a yellow solid. The solid was dissolved in DMSO (˜100 mg/mL) and purified by a single prep-HPLC purification (C₁₈, 5-μm, 50×250 mm, 75 mL/min, 80:20 to 59:41 0.3% HCO₂H (aq):MeCN over 9 minutes). Similarly, global supply of the antifungal caspofungin is supplied requiring only a single HPLC purification². Following HPLC purification, the solvent was removed in vacuo at 40° C. Upon complete solvent removal, residual formic acid was removed via azeotroping with milliQ water (10 mL) and toluene (50 mL). This process was repeated three times yielding AmBMU as a yellow solid (264.3 mg, 0.278 mmol, 64% average yield per step).

¹H NMR (750 MHz, 1:1 Pyridine d-5:Methanol d-4) δ 6.64 (dd, J=14.7, 11.2 Hz, 1H), 6.60 (dd, J=14.8, 10.0 Hz, 1H), 6.52 (t, J=12.1 Hz, 1H), 6.48-6.37 (m, 6H), 6.36-6.25 (m, 4H), 5.69-5.63 (m, 1H), 5.53-5.47 (dd, J=14.3, 9.8 Hz, 1H), 4.97 (s, 1H), 4.82-4.76 (m, 1H), 4.65 (app t, J=10.3 Hz, 1H), 4.53 (bs, 1H), 4.49 (app tt, J=9.8, 2.9 Hz, 1H), 4.42 (app t, J=9.1 Hz, 1H), 4.29-4.23 (m, 1H), 3.98 (app t, J=10.0 Hz, 1H), 3.90-3.84 (m, 2H), 3.80-3.72 (m, 1H), 3.62-3.56 (m, 1H), 3.56-3.51 (m, 1H), 3.47-3.44 (m, 1H), 3.38 (app d, J=9.5 Hz, 1H), 2.79 (s, 3H), 2.71-2.65 (m, 1H), 2.61-2.55 (m, 1H), 2.51 (dd, J=16.7, 9.8 Hz, 1H), 2.39-2.34 (m, 2H), 2.21-2.14 (m, 1H), 2.06-1.99 (m, 2H), 1.96 (dd, J=14.8, 7.3 Hz, 1H), 1.85 (dd, J=13.9, 10.9 Hz, 1H), 1.84-1.79 (m, 1H), 1.73-1.65 (m, 3H), 1.66-1.61 (m, 1H), 1.61-1.56 (m, 1H), 1.53 (app dt, J=14.0, 3.0 Hz, 1H), 1.47-1.45 (m, 1H), 1.46 (d, J=6.2 Hz, 3H), 1.37 (d, J=6.5 Hz, 3H), 1.25 (d, J=6.4 Hz, 3H), 1.18 (d, J=7.1 Hz, 3H).

¹³C NMR (125 MHz, 1:1 Pyridine d-5:Methanol d-4) δ 172.31, 161.63, 137.57, 137.33, 134.82, 134.20, 134.17, 133.99, 133.91, 133.67, 133.59, 133.28, 132.98, 131.04, 129.73, 128.99, 98.04, 98.26, 79.10, 77.44, 76.32, 75.24, 74.64, 72.32, 70.92, 70.45, 69.98, 69.26, 68.97, 68.69, 68.46, 58.35, 57.35, 47.22, 45.74, 44.90, 44.06, 42.89, 41.28, 40.80, 36.72, 36.38, 31.53, 27.02, 19.11, 18.10, 17.35, 12.64.

HRMS (ESI) Calculated (C₄₈H₇₇N₃O₁₆+H)⁺. 952.5382 Observed. 952.5385

Analytical HPLC (Zorbax Eclipse C₁₈, 1.8-μm, 2.1×50 mm, 0.4 mL/min, 95:5 to 5:95 H₂O:MeCN (both containing 0.1% HCO₂H) over 8 minutes) Retention Time=5.7 min.

### Example 2

### Synthesis of AmBAU from Minimally Protected AmB

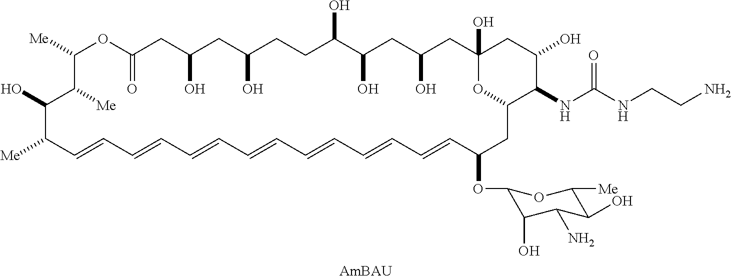 [SMILES: "AmBAU" ; CC1OC(O[C@H]2/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@]3(O)C[C@H](O)[C@@H](NC(=O)NCCN)[C@H](C2)O3)C(O)C(N)C1O]

A round bottom flask was charged with amphotericin B (1 g, ca. 1.082 mmol, 1 equiv.) and Fmoc-succinimide (0.55 g, 1.62 mmol, 1.5 equiv.) which were dissolved in a 2:1 mixture of DMF:MeOH (33.8 mL) at room temperature. Pyridine (0.5 mL, 6.21 mmol, 5.74 equiv.) was subsequently added and the reaction was stirred for 12 hours at room temperature. The reaction mixture was then poured into diethyl ether (1.0 L). After stirring for 30 minutes, the resulting yellow precipitate was isolated via Büchner filtration using Whatman #50 filter paper to afford a yellow solid. The filter cake was dried on the filter for 10 minutes and then stored under vacuum for one hour.

The resulting powder was dissolved in 1:1 THF:MeOH (35 mL) and cooled to 0° C. To this solution was added camphorsulfonic acid (138 mg, 0.59 mmol, 0.55 equiv.) and the resulting mixture was stirred for 1 hour at 0° C. The reaction was then quenched at 0° C. with triethylamine (0.14 mL, 0.59 mmol, 0.55 equiv.). The reaction was concentrated in vacuo removing approximately half of the solvent. The resulting saturated solution was poured into 1:1 hexanes:diethyl ether (1.0 L) and the yellow precipitate was collected via Büchner filtration using Whatman #50 filter paper and washed with diethyl ether (200 mL) to yield a yellow solid.

The resulting solid was dissolved in THF (54 mL, 0.02 M). To this solution was added triethylamine (0.15 mL, 1.08 mmol, 1 equiv.) and then diphenyl phosphoryl azide (0.70 mL, 3.25 mmol, 3 equiv.). The reaction was heated to 50° C. and stirred for 12 hours. After 12 hours, ethylene diamine (0.29 mL, 4.33 mmol, 4 equiv.) was added, and the reaction continued stirring at 50° C. for 3 hours, slowly evolving a yellow precipitate. The reaction mixture was then poured into diethyl ether (1.0 L), and the resulting yellow precipitate was isolated via Büchner filtration using Whatman #50 filter paper to afford a yellow solid which was dissolved in DMSO (˜66 mg/mL) and purified by prep-HPLC (C₁₈, 5-μm, 50×250 mm, 75 mL/min, 80:20 to 50:50 0.3% HCO₂H (aq):MeCN over 9 minutes). After HPLC purification the solvent was removed in vacuo at 40° C. Upon complete solvent removal, residual formic acid was removed via azeotroping with milliQ water (10 mL) and toluene (50 mL). This process was repeated three times yielding AmBAU as a yellow solid (236.2 mg, 0.241 mmol, 61% average yield per step).

¹H NMR (750 MHz, 1:1 Pyridine d-5:Methanol d-4) δ 6.64 (dd, J=14.7, 11.2 Hz, 1H), 6.60 (dd, J=15.2, 8.8 Hz, 1H), 6.55-6.47 (m, 1H), 6.47-6.35 (m, 7H), 6.35-6.25 (m, 3H), 5.68-5.61 (m, 1H), 5.49 (dd, J=15.0, 10.2 Hz, 1H), 4.91 (s, 1H), 4.79-4.73 (m, 1H), 4.64 (app t, J=10.7 Hz, 1H), 4.49-4.42 (m, 3H), 4.28-4.23 (m, 1H), 3.96 (app t, J=10.4 Hz, 1H), 3.88-3.82 (m, 1H), 3.82-3.73 (m, 3H), 3.55-3.50 (m, 1H), 3.51-3.46 (m, 1H), 3.47-3.43 (m, 1H), 3.39 (app d, J=8.8 Hz, 1H), 3.36 (app d, J=9.2 Hz, 1H), 3.32-3.26 (m, 1H), 3.23-3.16 (m, 1H), 2.66 (dd, J=15.4, 4.9 Hz, 1H), 2.59-2.53 (m, 1H), 2.49 (dd, J=16.8, 9.8 Hz, 1H), 2.38-2.33 (m, 2H), 2.18-2.12 (m, 1H), 2.04-1.97 (m, 2H), 1.90 (dd, J=14.9, 7.8 Hz, 1H), 1.84 (dd, J=14.0, 11.0 Hz, 1H), 1.82-1.75 (m, 1H), 1.71-1.65 (m, 3H), 1.65-1.60 (m, 1H), 1.60-1.55 (m, 1H), 1.52 (app dt, J=13.8, 2.9 Hz, 1H), 1.48-1.44 (m, 1H), 1.44 (d, J=6.2 Hz, 3H), 1.36 (d, J=6.5 Hz, 3H), 1.24 (d, J=6.4 Hz, 3H), 1.17 (d, J=7.1 Hz, 3H).

¹³C NMR (150 HMz, 1:1 Pyridine d-5:Methanol d-4) δ 172.34, 161.35, 137.58, 137.08, 134.78, 134.16, 134.11, 134.02, 133.93, 133.71, 133.62, 133.26, 133.00, 130.85, 130.62, 98.26, 98.07, 79.12, 77.60, 76.37, 75.24, 74.63, 72.28, 71.43, 70.47, 70.00, 69.23, 69.23, 68.69, 68.62, 58.16, 57.32, 47.18, 45.77, 44.93, 44.05, 42.86, 41.67, 40.80, 40.49, 39.31, 36.73, 36.35, 31.56, 19.08, 18.08, 17.32, 12.63.

HRMS (ESI) Calculated (C₄₉H₈₀N₄O₁₆+H)⁺. 981.5648 Observed. 981.5641

Analytical HPLC (Zorbax Eclipse C₁₈, 1.8-μm, 2.1×50 mm, 0.4 mL/min, 95:5 to 5:95 H₂O:MeCN (both containing 0.1% HCO₂H) over 8 minutes) Retention Time=5.2 min.

### Example 3

### Synthesis of AmBCU-Allyl Ester from Minimally Protected AmB

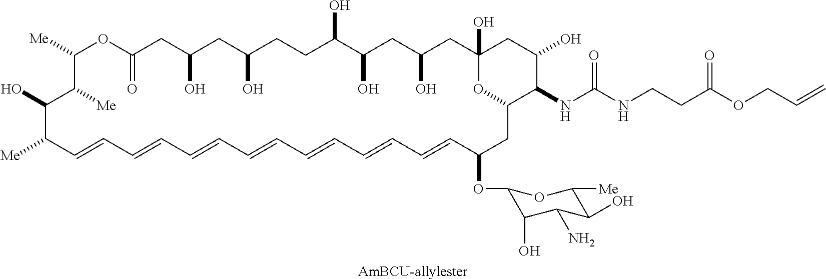 [SMILES: C=CCOC(=O)CCNC(=O)N[C@H]1[C@@H]2C[C@@H](OC3OC(C)C(O)C(N)C3O)/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@](O)(C[C@@H]1O)O2 ; "AmBCU-allylester"]

A round bottom flask was charged with amphotericin B (1 g, ca. 1.08 mmol, 1 equiv.) and Fmoc-succinimide (0.55 g, 1.62 mmol, 1.5 equiv.) which were dissolved in a mixture of 2:1 DMF:MeOH (33.8 mL) at room temperature. Pyridine (0.5 mL, 6.21 mmol, 5.74 equiv.) was subsequently added and the reaction was stirred for 12 hours at room temperature. The reaction mixture was then poured into diethyl ether (1.0 L). After stirring for 30 minutes, the resulting yellow precipitate was isolated via Büchner filtration using Whatman #50 filter paper to afford a yellow solid. The filter cake was dried on the filter for 10 minutes and then stored under vacuum for one hour.

The resulting powder was dissolved in 1:1 THF:MeOH (35 mL) and cooled to 0° C. To this solution was added camphorsulfonic acid (138 mg, 0.60 mmol, 0.55 equiv.) and the resulting mixture was stirred for 1 hour at 0° C. The reaction was then quenched at 0° C. with triethylamine (0.14 mL). The reaction was concentrated in vacuo removing approximately half of the solvent. The resulting saturated solution was poured into 1:1 hexanes:diethyl ether (1.0 L) and the yellow precipitate was collected via Büchner filtration using Whatman #50 filter paper and washed with diethyl ether (20 mL) to yield a yellow solid.

The resulting solid (1.06 g, ca. 1.08 mmol, 1 equiv.) was added to a 40 mL vial followed by THF (54 mL, 0.02M), triethylamine (0.16 mL, 1.14 mmol, 1.05 equiv.), and lastly diphenyl phosphoryl azide (0.70 mL, 3.25 mmol, 3 equiv.). The reaction was then heated to 50° C. and stirred for 15 hours.

To a separate 40 mL vial was added β-alanine allylester hydrochloride (7.16 g, 43.3 mmol, 40 equiv.), sodium carbonate (13.75 g, 129.8 mmol, 120 equiv.), and THF (14 mL). The resulting suspension stirred then at room temperature for 20 minutes. The suspension was then filtered through Celite followed by filtration through a syringe tip 0.2-μm filter. The resulting β-alanine allylester free base was then added to the 50° C. reaction mixture and allowed to stir for 8 hours. After 8 hours, the volatiles were removed in vacuo yielding a red oil. This was dissolved in DMSO and purified directly by prep HPLC (C₁₈, 5-μm, 50×250 mm, 80 mL/min, 80:20 to 40:60 0.3% HCO₂H (aq):MeCN over 9 minutes). Upon removal of the acetonitrile and aqueous formic acid solution in vacuo at 35° C., the C-13 methyl ketal is converted to a hemiketal yielding AmBCU-allylester as a yellow solid (370 mg, 0.352 mmol, 68% average yield per step).

¹H NMR (500 MHz, 10:1 Pyridine d-5:Methanol d-4) δ 6.71-6.25 (m, 13H), 6.01-5.89 (m, 1H), 5.70-5.64 (m, 1H), 5.54-5.48 (m, 1H), 5.33 (m, 1H), 5.20 (m, 1H), 4.97 (s, 1H), 4.79 (bs, 1H), 4.70-4.59 (m, 4H), 4.50 (app t, J=10.0 Hz, 1H), 4.43 (app t, J=8.8 Hz, 1H), 4.26-4.20 (m, 1H), 3.99 (app t, J=10.0 Hz, 1H), 3.88 (app d, J=10.8 Hz, 1H), 3.82-3.77 (m, 2H), 3.65-3.60 (m, 3H), 3.47 (m, 1H), 3.42-3.35 (m, 1H), 3.35-3.31 (m, 1H), 2.71-2.62 (m, 3H), 2.58 (m, 1H), 2.52 (dd, J =16.8, 9.7 Hz, 1H), 2.41-2.33 (m, 2H), 2.23-2.13 (m, 1H), 2.07-1.91 (m, 3H), 1.91-1.77 (m, 2H), 1.75-1.57 (m, 5H), 1.57-1.51 (m, 1H), 1.48-1.44 (m, 4H), 1.37 (d, J=6.3 Hz, 3H), 1.26 (d, J=6.3 Hz, 3H), 1.18 (d, J=7.1 Hz, 3H).

¹³C NMR (125 MHz, 10:1 Pyridine-d₅:MeOH-d₄) δ 172.31, 171.92, 160.41, 136.83, 136.75, 134.46, 133.84, 133.57, 133.47, 133.45, 133.20, 133.03, 132.91, 132.61, 130.57, 129.55, 117.73, 98.06, 97.90, 77.08, 75.96, 74.89, 74.31, 71.92, 71.61, 70.08, 69.59, 68.65, 68.29, 68.19, 65.31, 57.96, 57.09, 46.84, 45.53, 44.62, 42.59, 40.89, 40.48, 36.41, 36.07, 35.42, 31.22, 18.74, 17.89, 17.02, 12.32.

HRMS (ESI) Calculated (C₅₃H₈₃N₃O₁₈+H)⁺. 1050.570 Observed. 1050.5756.

Analytical HPLC (Zorbax Eclipse C₁₈, 1.8-μm, 2.1×50 mm, 0.4 mL/min, 95:5 to 5:95 H₂O:MeCN (both containing 0.1% HCO₂H) over 8 minutes) Retention Time=6.4 min.

### Example 4

### Synthesis of AmBCU via De-Allylation of AmBCU-Allyl Ester

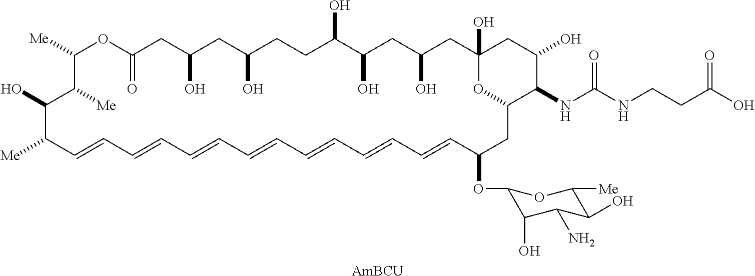 [SMILES: "AmBCU" ; CC1OC(O[C@H]2/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@]3(O)C[C@H](O)[C@@H](NC(=O)NCCC(=O)O)[C@H](C2)O3)C(O)C(N)C1O]

To a 40 mL vial was added AmBCU-allyl ester (370 mg, 352.3 μmol, 1 equiv.), and thiosalicylic acid (203.4 mg, 1.76 mmol, 5 equiv.). The vial was then brought into a glove box and Pd(PPh₃)₄ was added (205 mg, 0.18 mmol, 0.5 equiv.). The vial was sealed with a septa cap, removed from the glovebox, and DMF was added (17.6 mL, 0.2 M) via syringe. The reaction then stirred at room temperature for 1 hour. The reaction was then poured into Et₂O (370 mL) in multiple 50 mL centrifuge tubes. The resulting suspension was then centrifuged at 3700 G for 5 minutes. The pale red supernatant was decanted and the resulting yellow/orange solid was dissolved in DMSO and purified by prep HPLC (C₁₈, 5-μm, 50×250 mm, 80 mL/min, 80:20 to 40:60 0.3% HCO₂H (aq):MeCN over 9 minutes) yielding AmBCU as a yellow solid (124.4 mg, 0.123 mmol, 35% yield, 58% average yield per step from 1 g AmB).

¹H NMR (500 MHz, 1:1 Pyridine d-5:Methanol d-4) δ 6.69-6.23 (m, 13H), 5.70-5.64 (m, 1H), 5.54-5.50 (m, 1H), 5.00 (s, 1H), 4.76 (bs, 1H), 4.65 (app t, J=10.4 Hz, 1H), 4.57 (app bs, 1H), 4.49 (app t, J=9.0 Hz, 1H), 4.45-4.39 (m, 1H), 4.30-4.23 (m, 1H), 4.01-3.95 (m, 1H), 3.92-3.84 (m, 2H), 3.82-3.77 (m, 1H), 3.73-3.67 (m, 2H), 3.67-3.63 (m, 2H), 3.57-3.51 (m, 2H), 3.49-3.45 (m, 1H), 3.38 (app d, J=9.8 Hz, 1H), 2.74-2.63 (m, 2H), 2.51 (dd, J=17.0, 9.5 Hz 1H), 2.43-2.34 (m, 2H), 2.23-2.14 (m, 1H), 2.05-1.98 (m, 2H), 2.94-1.89 (m, 1H), 1.88-1.80 (m, 2H), 1.73-1.59 (m, 5H), 1.57-1.51 (m, 1H), 1.46 (d, J=6.1 Hz, 4H), 1.44-1.40 (m, 1H), 1.38 (d, J=6.3 Hz, 3H), 1.26 (d, J=6.3 Hz, 3H), 1.19 (d, J=7.1 Hz, 3H).

¹³C NMR (150 MHz, DMSO-d₆) δ 175.38, 170.58, 158.41, 136.81, 136.33, 133.92, 133.76, 133.64, 133.25, 133.16, 132.57, 132.40, 132.25, 132.17, 131.87, 131.23, 130.28, 129.00, 97.04, 96.87, 77.20, 76.06, 73.86, 73.48, 73.05, 69.50, 69.16, 68.80, 67.98, 67.79, 67.44, 66.84, 66.20, 59.75, 56.23, 55.25, 45.84, 44.88, 44.80, 42.52, 42.01, 40.43, 39.52, 35.11, 30.96, 29.05, 18.52, 17.80, 16.96, 12.10.

HRMS (ESI) Calculated (C₅₀H₇₉N₃O₁₈+H)⁺ 1010.5437 Found. 1010.5449.

Analytical HPLC (Zorbax Eclipse C₁₈, 1.8-μm, 2.1×50 mm, 0.4 mL/min, 95:5 to 5:95 H₂O:MeCN (both containing 0.1% HCO₂H) over 8 minutes) Retention Time=6.07 min.

### Example 5

### Persilyl AmB Intermediate 4

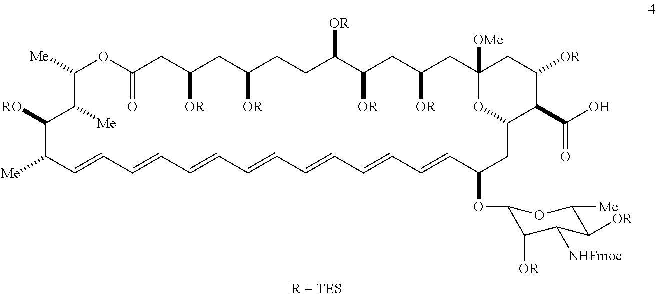

Persilyl intermediate 4 was synthesized as depicted in FIG. 4, following a protocol found in: Palacios, D. S., Anderson, T. M. & Burke, M. D. A Post-PKS Oxidation of the Amphotericin B Skeleton Predicted to be Critical for Channel Formation Is Not Required for Potent Antifungal Activity. J. Am. Chem. Soc. 129, 13804-13805, (2007).

### Example 6

### Syntheses of Isocyanate 5

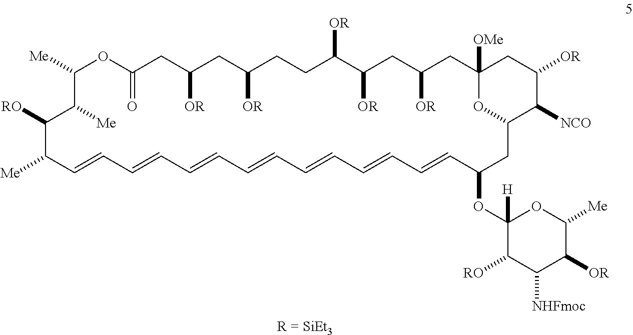

To a 40 mL vial was added 4 (602.6 mg, 275.3 μmol, 1 equiv.), and benzene (13.7 mL). Triethyl amine (115 μL, 0.822 mmol, 3 equiv.) was added followed by DPPA (71 μL, 33.0 mmol, 1.2 equiv.). The reaction was then placed in a preheated heating block at 80° C. and allowed to stir for 3.5 hours. The reaction was then transferred to a 125 mL separatory funnel with water (25 mL) and diethyl ether (50 mL). The layers were separated and the organic layer was washed with brine (25 mL), dried over Na₂SO₄, filtered and concentrated in vacuo. The resulting red/orange oil was then purified by SiO₂ chromatography (100:0 to 0:100 Hexane:Et₂O) yielding 5 as an orange solid (168.7 mg, 0.077 mmol, 28% yield).

¹H NMR (500 MHz, Acetone-d₆) δ 7.87 (d, J =7.5 Hz, 2H), 7.70 (d, J =7.5 Hz, 2H), 7.42 (t, J =7.4 Hz, 2H), 7.37-7.29 (m, 2H), 6.59-6.11 (m, 12H), 6.06 (dd, J =15.6, 5.9 Hz, 1H), 5.51 (dd, J=14.8, 9.4 Hz, 1H), 5.36 (d, J=9.7 Hz, 1H), 4.70 (s, 1H), 4.69-4.64 (m, 2H), 4.48 (dd, J=10.5, 6.5 Hz, 1H), 4.35 (dd, J=10.5, 6.6 Hz, 1H), 4.24 (app t, J=6.6 Hz, 1H), 4.18 (s, 1H), 4.16-4.05 (m, 2H), 4.04-3.98 (m, 1H), 3.97 (app d, J=3.1 Hz, 1H), 3.86 (app dd, J=9.0, 2.9 Hz, 1H), 3.80 (app t, J=9.4 Hz, 1H), 3.73-3.60 (m, 2H), 3.48 (app t, J=9.0 Hz, 1H), 3.38-3.31 (m, 1H), 3.29 (app t, J=9.7 Hz, 1H), 3.16 (s, 3H), 2.48-2.41 (m, 1H), 2.38 (dd, J=14.8, 6.2 Hz, 1H), 2.20-2.14 (m, 1H), 1.96-1.87 (m, 2H), 1.87-1.81 (m, 4H), 1.69-1.60 (m, 3H), 1.57-1.47 (m, 1H), 1.29 (s, 4H), 1.25 (d, J=6.2 Hz, 3H), 1.18 (d, J=6.0 Hz, 3H), 1.10-0.88 (m, 89H), 0.80-0.50 (m, 54H).

IR (thin film, cm⁻¹) 2956.35, 2912.00, 2877.29, 2250.53, 1731.77, 1590.99, 1488.78, 1457.93, 1415.50, 1378.86, 1303.65, 1272.79, 1238.08, 1205.29, 1184.08, 1160.94, 1108.87, 1076.09, 1008.59, 966.16, 738.60.

TLC (7:3 Hexanes:Et₂O, CAM stain) R_{f}=0.64

HRMS (ESI) Calculated (C₁₁₇H₂₁₀N₂O₁₈Si₉+Na)⁺ 2206.3400 Found. 2206.3413.

In a separate synthesis, to a 50 mL round-bottom flask was added 4 (781 mg, 357.2 μmol, 1 equiv.) in benzene (18 mL). Triethyl amine (150 μL, 1.07 mmol, 3 equiv.) was added slowly followed by dropwise addition of DPPA (92 μL, 0.429 mmol, 1.2 equiv.). The reaction was then heated at 85° C. in an oil bath and reaction progress monitored by TLC. After 5 h, starting material was fully consumed and the reaction was cooled 0° C. and slowly poured into a mixture of water (25 mL) and diethyl ether (50 mL) cooled to 0° C. The mixture was then transferred to a separatory funnel and the aqueous layer extracted with diethyl ether (25 mL×3). The combined organic layers were washed with brine (50 mL), dried over MgSO₄, filtered, and concentrated in vacuo. The resulting red oil was loaded onto a normal-phase silica column as a solution in benzene and purified by SiO₂ chromatography (19:1 to 3:1 heptane:Et₂O) yielding 5 as a yellow solid (158 mg, 0.0724 mmol, 20% yield).

¹H NMR (500 MHz, Acetone-d₆) δ 7.88 (d, J=7.6 Hz, 2H), 7.70 (d, J=7.5 Hz, 2H), 7.43 (t, J=7.5 Hz, 2H), 7.38-7.28 (m, 2H), 6.61-5.99 (m, 11H), 4.69 (d, J=15.2 Hz, 2H), 4.24 (t, J=6.6 Hz, 2H), 4.09 (s, 2H), 4.01 (s, 1H), 3.86 (d, J=7.4 Hz, 1H), 3.80 (t, J=9.4 Hz, 1H), 3.74-3.55 (m, 3H), 3.35 (t, J=7.2 Hz, 1H), 3.32-3.21 (m, 1H), 3.16 (s, 2H), 2.60 (d, J=6.5 Hz, 2H), 2.43 (dt, J=15.1, 7.7 Hz, 2H), 1.87-1.60 (m, 7H), 1.27 (dq, J=14.2, 7.9 Hz, 5H), 1.18 (d, J=6.1 Hz, 3H), 1.12-0.82 (m, 73H), 0.82-0.50 (m, 46H).

IR (thin film): ν 2955, 2911, 2877, 2248, 1736, 1460, 1110, 1005 cm⁻¹.

### Example 7

### Synthesis of Carbonate 6

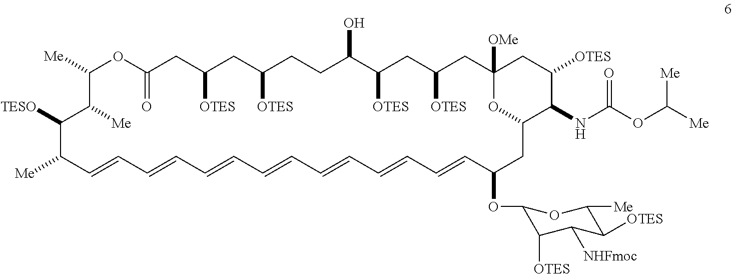

To a 1.5 mL vial was added 5 (as a stock solution (100 μL of 150 mg in 1.5 mL benzene) 10 mg, 4.57 μmol, 1 equiv.), and Titanium isopropoxide (as a stock solution (50 μL of 25 μL in 4.6 mL benzene) 0.27 μL, 0.914 μmol, 0.2 equiv.) and THF (80 μL). The reaction was then allowed to stir at room temperature for 1 hour. The reaction was then diluted with water (1.5 mL) and diethyl ether (1.5 mL). The layers were separated and the organic layer was dried over Na₂SO₄, filtered and concentrated in vacuo. The resulting red/orange oil was then purified by SiO₂ chromatography (100:0 to 80:20 Hexane:Et₂O) yielding 5 as an orange solid.

¹H NMR (500 MHz, Acetone-d₆) δ 7.88 (d, J=7.5 Hz, 2H), 7.70 (d, J=7.5 Hz, 2H), 7.43 (t, J=7.3 Hz, 2H), 7.36-7.32 (m, 2H), 6.59-6.08 (m, 12H), 6.03 (dd, J=15.5, 6.1 Hz, 1H), 5.51 (dd, J=14.9, 9.5 Hz, 1H), 5.35 (d, J=9.9 Hz, 1H), 4.87 (p, J=6.3 Hz, 1H), 4.77-4.73 (m, 1H), 4.71-4.67 (m, 1H), 4.65 (s, 1H), 4.48 (dd, J=10.5, 6.5 Hz, 1H), 4.37 (dd, J=10.4, 6.5 Hz, 1H), 4.25 (app t, J=6.3 Hz, 2H), 4.18-4.09 (m, 1H 4.07-3.97 (m, 2H), 3.87-3.84 (m, 1H), 3.76 (app dd, J=11.8, 6.9 Hz, 1H), 3.70 (d, J=8.9 Hz, 1H), 3.74-3.66 (m, 2H), 3.47-3.34 (m, 2H), 3.35-3.28 (m, 1H), 3.15 (s, 3H), 2.58 (d, J=6.6 Hz, 1H), 2.47-2.40 (m, 2H), 2.26 (app dd, J=15.6, 7.4 Hz, 2H), 2.20-2.15 (m, 1H), 1.94-1.85 (m, 4H), 1.84-1.80 (d, J=13.1 Hz, 3H), 1.79-1.68 (m, 4H), 1.68-1.61 (d, J=9.3 Hz, 2H), 1.54-1.56 (s, 1H), 1.26 (app dd, J=6.2, 3.2 Hz, 6H), 1.22 (d, J=6.2 Hz, 3H), 1.18 (d, J=6.0 Hz, 3H), 1.14-0.83 (m, 87H), 0.81-0.53 (m, 54H).

LRMS (ESI) Calculated (C₁₂₀H₂₁₈N₂O₁₉Si₉+Na)⁺ 2266.4 Found. 2266.6.

TLC (7:3 Hexanes:Et₂O, CAM stain) R_{f}=0.51

### Example 8

### Synthesis of Carbonate 7

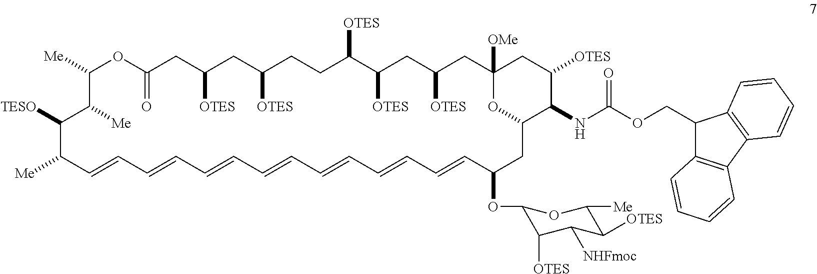

To a 1.5 mL vial was added 5 (as a stock solution (100 μL of 150 mg in 1.5 mL benzene) 10 mg, 4.57 μmol, 1 equiv.), Titanium tertbutoxide (as a stock solution (50 μL of 25 μL in 3.5 mL benzene) 0.35 μL, 0.914 μmol, 0.2 equiv.), and Fmoc-alcohol (as a stock solution (21.1 mg in 1.58 mL) 1.33 mg, 9.15 μmol, 2 equiv.). The reaction was then allowed to stir at room temperature for 1 hour. The reaction was then diluted with water (1.5 mL) and diethyl ether (1.5 mL). The layers were separated and the organic layer was dried over Na₂SO₄, filtered and concentrated in vacuo. The resulting red/orange oil was then purified by SiO₂ chromatography (100:0 to 80:20 Hexane:Et₂O) yielding 7 as an orange solid.

LRMS (ESI) Calculated (C₁₂₀H₂₁₈N₂O₁₉Si₉+Na)⁺ 2402.4 Found. 2402.5.

TLC (7:3 Hexanes:Et₂O, CAM stain) R_{f}=0.53

### Example 9

### Synthesis of Amide 8

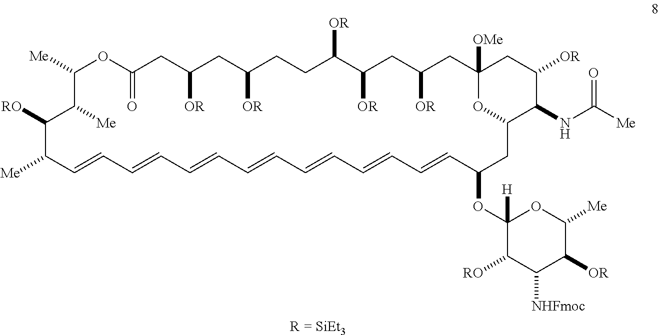

To a dry 20 mL vial was added 5 (100 mg, 45.8 mmol, 1 equiv.) in THF (2.3 mL). The reaction vessel was cooled to 0° C. and Me₂Zn (48 μL of a 0.85 M solution in THF, 0.041 mmol, 0.9 equiv.) was added dropwise to the reaction. The reaction was allowed to stir at 0° C. for 1 h and then additional Me₂Zn (48 μL of a 0.85 M solution in THF, 0.041 mmol, 0.9 equiv.) was added. The reaction was stirred at 0° C. for 1 h and then quenched at 0° C. by dropwise addition of H₂O (5 mL). The mixture was warmed to room temperature, transferred to a separatory funnel, and extracted with diethyl ether (10 mL×3). The combined organic layers were washed with brine (10 mL), dried over MgSO₄, filtered, and concentrated in vacuo. The resulting oil was loaded onto a normal-phase silica column as a solution in benzene and purified by SiO₂ chromatography (100:0 to 3:2 hexanes:Et₂O) yielding 8 as a yellow solid (40 mg, 0.018 mmol, 40%).

¹H NMR (500 MHz, Acetone-d₆) δ 7.90 (d, J=7.6 Hz, 2H), 7.72 (d, J=7.5 Hz, 2H), 7.45 (t, J=7.5 Hz, 2H), 7.38 (s, 6H), 6.55 (td, J=17.4, 16.3, 9.9 Hz, 3H), 6.49-5.96 (m, 11H), 5.64 (s, 5H), 4.71 (d, J=16.1 Hz, 3H), 4.26 (q, J=7.5, 7.0 Hz, 2H), 4.20-4.07 (m, 2H), 3.88 (dd, J=9.0, 2.8 Hz, 1H), 3.82 (t, J=9.4 Hz, 1H), 3.77-3.60 (m, 3H), 3.50 (t, J=8.9 Hz, 1H), 3.43 (q, J=7.0 Hz, 4H), 3.40-3.35 (m, 1H), 3.33 (d, J=5.4 Hz, 2H), 3.31 (s, 1H), 3.19 (s, 3H), 2.62 (d, J=6.5 Hz, 2H), 1.98-1.92 (m, 2H), 1.85 (s, 2H), 1.84-1.72 (m, 5H), 1.72-1.61 (m, 2H), 1.38-1.23 (m, 13H), 1.20 (d, J=6.0 Hz, 4H), 1.13 (t, J=7.0 Hz, 7H), 1.11-0.85 (m, 110H), 0.85-0.54 (m, 66H).

### Example 10

### In Vitro Assessment of Biological Activity

Each derivative proposed herein is tested for biological activity against both yeast and human cells to determine its therapeutic index. A broth microdilution experiment determines the MIC (minimum inhibitory concentration) of each derivative against S. cerevisiae and the clinically relevant C. albicans, thereby establishing the antifungal activity of each novel derivative. To test for toxicity against human cells, each compound is exposed to a hemolysis assay against red blood cells which determines the concentration required to cause 90% lysis of human red blood cells (EH₉₀). Additionally, each compound is exposed to human primary renal tubule cells to determine the toxicity of each compound against kidney cells. These assays when compared against the known values of AmB against the same cell lines determine the improvement in therapeutic index of each compound.

### Example 11

### In Vivo Assessment of Biological Activity

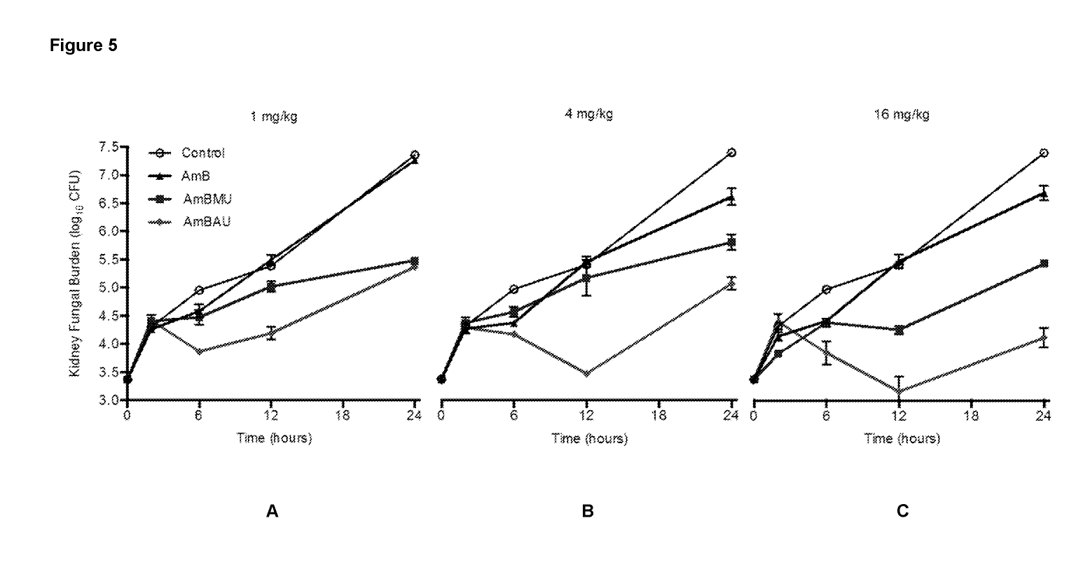

The antifungal efficacies of AmBMU and AmBAU were tested in a mouse model of disseminated candidiasis. In this experiment neutropenic mice were infected with C. albicans via the tail vein, and then 2 hours post infection the mice were treated with a single intraperitoneal injection of AmB, AmBMU, or AmBAU. Then 2, 6, 12, and 24 hours post infection the mice were sacrificed, and the fungal burden present in their kidneys was quantified. Results are shown in FIG. 5. Both AmBMU and AmBAU were substantially more effective than AmB at reducing the fungal burden present in the kidneys at all three tested doses (i.e., 1, 4, and 16 mg/kg). The differences were most pronounced at the 16 mg/kg dose at 24 hours post inoculation. Relative to AmB, AmBMU reduced the fungal burden by 1.2 log units (p≤0.001), and AmBAU reduced the fungal burden by nearly 3 log units (p≤0.0001). We speculate that an improved pharmacological profile, potentially due to greatly increased water solubility, may contribute to the unexpected and dramatic improvements in in vivo antifungal activity for the new compounds.

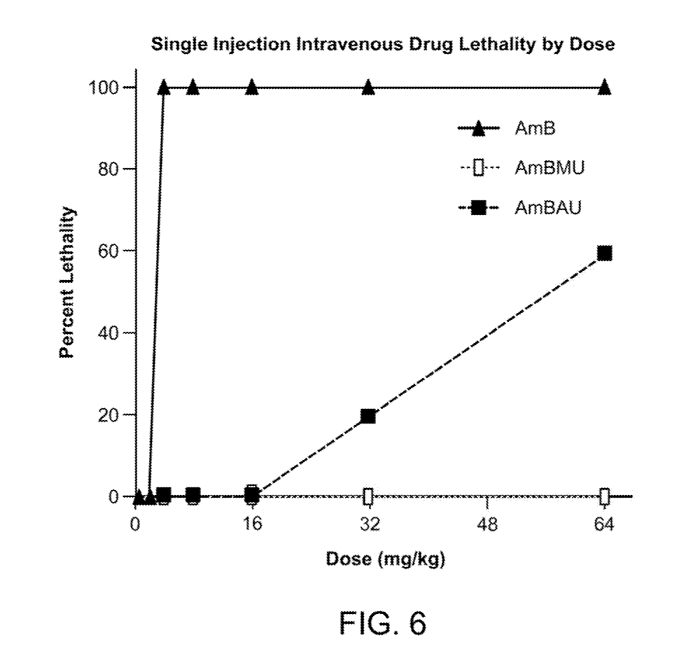

In a separate set of experiments, acute toxicity was evaluated by single intravenous administration of 1, 2, 4, 8, 16, 32, or 64 mg/kg AmB or its derivatives to healthy mice, followed by monitoring for lethality. Results are shown in FIG. 6. All mice in the 4 mg/kg AmB dosage group died within seconds. AmBAU was drastically less toxic with >50% lethality not being reached until the 64 mg/kg dosage group. Strikingly, all mice dosed with 64 mg/kg AmBMU survived with no observable toxicity.

1. A compound represented by formula (I): 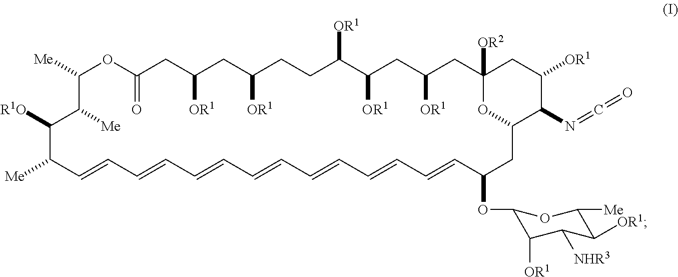
or a pharmaceutically acceptable salt thereof;
wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

2. The compound of claim 1, wherein R¹ represents trialkylsilyl.

3-4. (canceled)

5. The compound of claim 1 wherein R³ represents (aralkyl)OC(O)—.

6. (canceled)

7. A compound represented by formula (II): 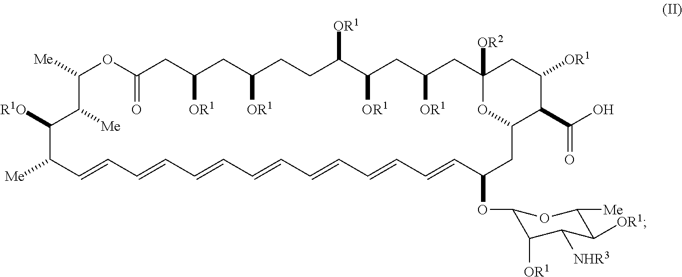
or a pharmaceutically acceptable salt thereof;
wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl; and
R³ represents (alkyl)OC(O)—, (aralkyl)OC(O)—, (alkenyl)OC(O)—, (cycloalkyl)OC(O)—, or (haloalkyl)OC(O)—.

8. The compound of claim 7, wherein R¹ represents trialkylsilyl.

9-10. (canceled)

11. The compound of claim 7, wherein R³ represents (aralkyl)OC(O)—.

12. (canceled)

13. A compound according to formula: 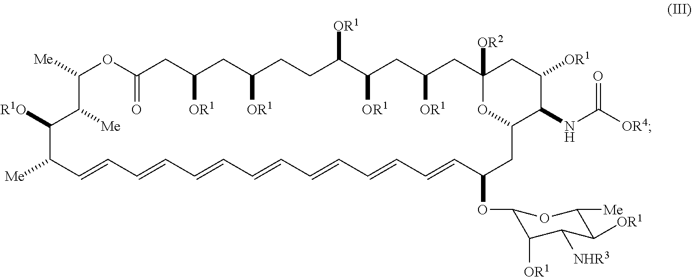
or a pharmaceutically acceptable salt thereof;
wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—; and
R⁴ represents alkyl, aralkyl, alkenyl, aryl, or cycloalkyl.

14. The compound of claim 13, wherein R¹ represents trialkylsilyl.

15-16. (canceled)

17. The compound of claim 13, wherein R³ represents (aralkyl)OC(O)—.

18. (canceled)

19. The compound of claim 13, wherein R⁴ represents isopropyl or 9-fluorenylmethyl.

20. AmBCU-allylester, or a pharmaceutically acceptable salt thereof:

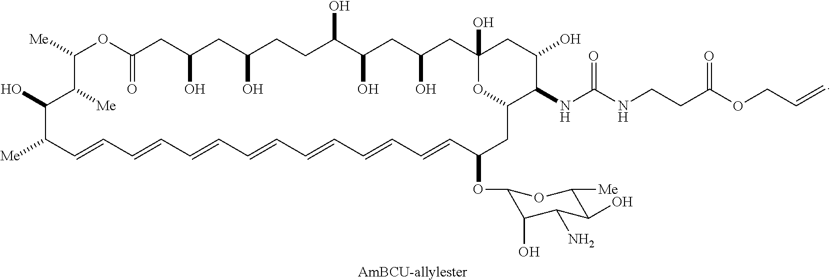 [SMILES: C=CCOC(=O)CCNC(=O)N[C@H]1[C@@H]2C[C@@H](OC3OC(C)C(O)C(N)C3O)/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@](O)(C[C@@H]1O)O2 ; "AmBCU-allylester"]

21. A method of preparing a compound of formula (IV), or a pharmaceutically acceptable salt thereof: 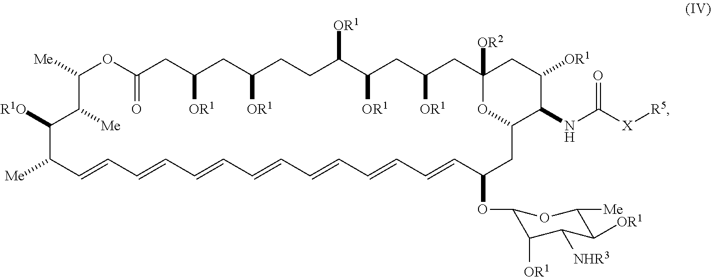
comprising the step of combining a compound of formula (I) or a pharmaceutically acceptable salt thereof, and a compound represented by R⁵-XH or R⁵-X-M, thereby producing the compound of formula (IV);
wherein:
R¹ represents, independently for each occurrence, trialkylsilyl, dialkylarylsilyl, alkyldiarylsilyl, triarylsilyl, (alkyl)OCH₂—, (alkenyl)OCH₂—, (aralkyl)OCH₂—, (alkoxyalkyl)OCH₂—, (aryl)OCH₂—, or ((trialkylsilyl)alkyl)OCH₂—;
R² represents (C₁-C₆)alkyl;
R³ represents (alkyl)OC(O)—, (aralkyl)OC(O)—, (alkenyl)OC(O)—, (cycloalkyl)OC(O)—, or (haloalkyl)OC(O)—;
X represents O, NH, or N(R⁶); and
R⁵ and R⁶ each independently represent alkyl, aralkyl, alkenyl, aryl, or cycloalkyl;
the compound of formula (I) is represented by: 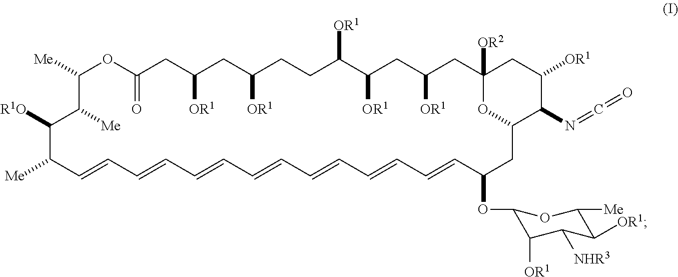
and
M is an alkali metal cation, an alkaline earth cation, or a transition metal cation.

22. The method of claim 21, further comprising the step of combining a compound of formula (II), and a phosphoryl azide, thereby producing the compound of formula (I);
wherein the compound of formula (II) is represented by:

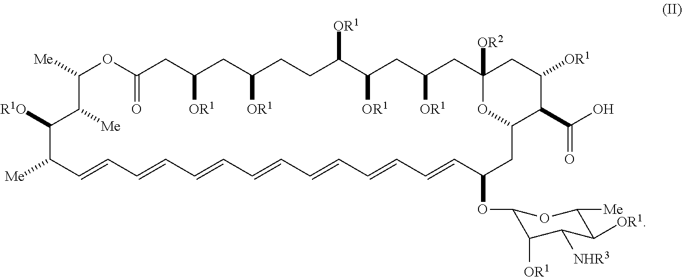

23. The method of claim 22, wherein the phosphoryl azide is diphenyl phosphoryl azide.

24. The method of claim 21, wherein X is O;
and M is Na, K, or Ti(OR⁵)₃.

25. The method of claim 21, wherein the step of combining a compound of formula (I) or a pharmaceutically acceptable salt thereof and a compound represented by R⁵-XH or R⁵-X-M further comprises a Lewis acid.

26. (canceled)

27. The method of claim 22, wherein the step of combining a compound of formula (II) and a phosphoryl azide further comprises a Bronsted base.

28. (canceled)

29. The method of claim 21, wherein R¹ represents trialkylsilyl.

30. (canceled)

31. The method of claim 21, wherein R² represents methyl.

32. The method of claim 21, wherein R³ represents (aralkyl)OC(O)—.

33. (canceled)
